(19) United States
(12) Patent Application Publication
(10) Pub. No.: US 2008/0214903 A1
(43) Pub. Date: Sep. 4, 2008

(54) Methods and Systems for Physiological and Psycho-Physiological Monitoring and Uses Thereof

(76) Inventor: Tuvi Orbach, London (GB)

Correspondence Address: NATH & ASSOCIATES, 112 South West Street, Alexandria, VA, 22314, US

(21) Appl. No.: 11/884,775

(22) PCT Filed: Feb. 22, 2006
(86) PCT No.: PCT/IL2006/000230

§ 371 Date: Feb. 21, 2008

Related U.S. Application Data
(60) Provisional application No. 60/654,460, filed on Feb. 22, 2005.

(51) Int. Cl. A61B 5/00; G06Q 50/00

(52) U.S. Cl. 600/301; 705/2

(57) ABSTRACT

The invention provides a system and method for monitoring one or more physiological parameters of a user. The system of the invention includes one or more wearable sensor modules sensing the one or more physiological parameters. One or more transmitters wirelessly transmit signals indicative of values of the one or more physiological parameters to a mobile monitor. The mobile monitor includes a processor processing the signals received from the transmitter in real time using expert knowledge. A device provides one or more indications of results of the processing. The invention also provides wearable mobile sensors for use in the system of the invention. The method of the invention includes obtaining values of the physiological parameters of the user from one or more wearable sensor modules. Signals indicative of values of the one or more physiological parameters are wirelessly transmitted to a mobile monitor. The signals are processed in real time using expert knowledge, and one or more indications of results of the processing are provided to the mobile unit.

### FIELD OF THE INVENTION

The present invention is related generally to the field of physiologically monitoring and bio interactive applications.

### BACKGROUND OF THE INVENTION

Biofeedback has been in use for many years to alleviate and change an individual's negative behavior patterns but existing systems have a number of significant drawbacks: Most current systems are reliant on powerful computers First of all, they require the user to be trained either by health professionals or complex on-line programmers. Once the user has been trained, they must remember to implement the internal physiological changes in their daily lives. The biofeedback sessions are rarely undertaken on a daily basis and certainly not in real time. This requires the user to remember specific events that occurred days before and recall his exact emotional responses.

U.S. Pat. No. 6,026,322; entitled “Biofeedback apparatus for use in therapy”; to Korenman, et al. filed Feb. 6, 1997; discloses an apparatus and a program designed to train the user to control one or more aspects of his or her psycho-physiological state by controlling signals representative of a psycho-physiological parameter of the user, e.g. his galvanic skin resistance, which may be detected by a sensor unit with two contacts on adjacent fingers of a user. The sensor unit can be separate from a receiver unit which is connected to a computer running the program. The disclosed apparatus is described for use in treating patients with a physiological condition, for example, irritable bowel syndrome. In a treatment session, one or more psycho-physiological parameters of the patient are sensed and the sensed parameter used to alter a display which the patient watches. The display includes a visual or pictorial representation of the physiological condition being treated which changes in appearance in a fashion corresponding to the physiological change desired in the patient.

PCT application WO0047110; discloses a method for obtaining continuously and non-invasively one or more parameters relating to the cardiovascular system of a subject, for example: systolic blood pressure, diastolic blood pressure, young modulus of an artery, cardiac output, relative changes in vascular resistance, and relative changes in vascular compliance.

U.S. Pat. No. 6,067,468; to Korenman, discloses a program, designed to train the user to control one or more aspects of his or her psycho-physiological state. The program is controlled by signals representative of a psycho-physiological parameter of the user, e.g., his galvanic skin resistance which may be detected by a sensor unit with two contacts on adjacent fingers of a user. The sensor unit is separate from a receiver unit which is connected to a computer running the program.

### SUMMARY OF THE INVENTION

In its first aspect, the present invention provides portable, cordless, and wearable sensors, for monitoring queries emotional and physiological responses to events as they occur. These results, gathered in real time, may be more effective and relevant to the user than those recreated days later after they occurred, under artificial conditions. The new sensors may utilize mobile phones and other technology to display the user's physiology and emotional state, real-time coaching based on expert knowledge, and to train the user to modify negative behavior patterns.

As used herein, the term “wearable device” refers to a device that the user can carry with him, for example, under or above his clothing, in his pocket, attached to his clothes, or in his hand.

In its second aspect, the invention provides a system for monitoring a user's emotional and physiological responses to events as they occur.

In another of its aspects, the invention provides methods to analyze the user's state of mind and physiology. In yet another of its aspects, the invention provides applications of the methods and sensors of the invention.

The invention also provides new methods to assess subtle information from this data—such as the user's emotions; new methods of therapy; and new methods of entertainment, based on the interactions with the user's physiology and responses.
Thus, in one of its aspects, the invention provides a system for monitoring one or more physiological parameters of a user comprising:
   (a) one or more wearable sensor modules sensing the one or more physiological parameters;
   (b) one or more transmitters wirelessly transmitting first signals indicative of values of the one or more physiological parameters to a mobile monitor; and
   (c) the mobile monitor, wherein the mobile monitor comprises:
      a first processor processing the first signals received from the transmitter in real time using expert knowledge; and
      a device providing one or more indications of results of the processing.
   The system of the invention may further comprise a remote server capable of communication with said mobile monitor, the remote server receiving second signals from the mobile monitor, the remote server associated with a viewing station having a second processor, the remote server being configured to perform at least one of the following:
   (a) transmitting the second signals to a viewing station for analysis, the analysis;
   (b) accessing historical data relating to the subject;
   (c) transmitting the historical data to the viewing station;
   (d) receiving from the viewing station results of the analysis;
   (e) transmitting the results of the analysis to the mobile unit; the analysis being based upon the second signals, and one or more of the historical data, expert knowledge and computerised protocols.
   At least one sensor module of the system may comprise at least one sensor selected, for example, from the group comprising:
   (a) An electro dermal activity sensor;
   (b) An electrocardiogram sensor;
   (c) A plethysmograph; and
   (d) A piezoelectric sensor.
   The system of the invention may comprise at least two sensors selected, for example, from a group comprising:
   (a) an electro dermal activity sensor;
   (b) an electrocardiogram sensor;
   (c) a plethysmograph; and
   (d) a respiration sensor.
The first signals may be transmitted from a sensor module to the mobile monitor, for example, by any one or more of the following protocols:
   (a) Bluetooth;
   (b) WiFi; and
   (c) Wireless Lan;
   The mobile monitor may be selected, for example, from the group comprising:
   (a) a cellular phone;
   (b) a personal digital assistant (PDA);
   (c) a pocket PC;
   (d) a mobile audio digital player;
   (e) an iPod,
   (f) an electronic note-book;
   (g) a personal laptop computer;
   (h) a DVD player;
   (i) a hand held video game with wireless communication; and
   (j) a mobile TV.
   The mobile unit may be a cellular telephone and communication between the mobile monitor and the remote server may be over a cellular communication network.
   The mobile unit may include any one or more of a visual display, one or more speakers, a headphone, and a virtual reality headset.
   In another of its aspects, the invention provides a wearable sensor module for use in the system of the invention.
   The wearable sensor module may comprise at least one sensor selected, for example, from the group comprising:
   (a) An electro dermal activity sensor;
   (b) An electrocardiogram sensor;
   (c) A plethysmograph; and
   (d) A pizoomagnetic sensor.
   The wearable sensor module may comprise at least two sensors selected, for example, from a group comprising:
   (a) an electro dermal activity sensor;
   (b) an electrocardiogram sensor;
   (c) a plethysmograph; and
   (d) a respiration sensor.
   The wearable sensor module may comprise a transmitter transmitting signals, for example, by any one or more of the following protocols:
   (a) Bluetooth;
   (b) WiFi; and
   (c) Wireless Lan;
   The wearable sensor unit may comprise an electro dermal activity sensor adapted to monitor skin conductivities using at least a 16 bit A to D conversion without the need of manual calibration.
   The sensor module may comprise an EDA sensor comprising:
   (a) at least two electrodes adapted to be applied to a skin surface;
   (b) electronic circuitry for measuring a skin resistance across the electrodes and calculating an EDA based upon the resistance using an algorithm in which the EDA does not depend linearly on the resistance.
   The sensor module may comprise a blood flow sensor comprising:
   (a) a light source adapted to emit light towards a skin surface;
   (b) a light detector adapted to detecting light reflected from the skin surface;
   (c) electronic circuitry for measuring an intensity of the reflected light and controlling an intensity of said light source based upon the intensity of the reflected light.
   Electronic circuitry in the sensor module may be capable of measuring skin resistance across the electrodes over a range of at least from 50 K Ohm to 12 M Ohm.
   The first processor of the system of the invention may be configured to calculate from the first signals one or both of a parameter indicative of an arousal state of the user and a parameter indicative of an emotional state of the user.
   Calculation of a parameter indicative of an arousal state of the user may include calculating a score of a sympathetic and parasympathetic activity of the user using an algorithm based on any one or more of the user's Electro Dermal activity, Heart Rate, EDA variability, and HR variability.
   The first processor may be configured to calculate a parameter indicative of an arousal state of the user and to display the parameter indicative of an arousal state of the user on a display associated with the mobile unit as a two-dimensional vector.
   The first processor may be configured to display on a display associated with the mobile monitor any one or more of the following images: an image indicative of bio-feedback information relating to the user; an image indicative of breathing activity of the user, an image including a graph indicative of an EDA activity of the user, an image including a graph indicative of a heart rate of the user, an image including a graph indicative of a heart rate variability of the user; an image including a graph indicative of an autocorrelation of a heart rate variability of the user; and an image indicative of recommendation to improve the user's psycho-physiological state based on one or both of the user's physiological data and experts' knowledge.
   An image indicative of breathing activity may include a bar having a length indicative of the breathing activity. An image indicative of bio-feedback information relating to the user may include one or more parameter target values.
   The first processor may be configured to calculate in a calculation based upon the first signals any one or more of the following: a breathing rate of the user; and a heart rate variability of the user. The user's rate of breathing may be calculated and analysis by monitoring changes in the electrical capacitance of the body while the user is breathing.
   The system of the invention may further comprise an entertainment system In this case, the first processor may be configured to determine at least one command based on the first signals and to transmit the at least one command based to the entertainment system. The entertainment system may comprise a third processor configured to perform an action based upon the one or more commands. The action may comprises any one or more of generating an SMS massage, controlling a DVD, controlling a computer game, and controlling a “Tamaguchi” animation. The action may comprise processing a user reaction to any one or more of the following: a displayed animated image; a video clip, an audio clip, a multimedia presentation, real-time communication with another human, a question that the user has to answer, and a task that the has to perform.
   In another of its aspects, the invention provides a method for monitoring one or more physiological parameters of a user comprising:
   (a) obtaining values of the physiological parameters of the user from one or more wearable sensor modules;
   (b) wirelessly transmitting first signals indicative of values of the one or more physiological parameters to a mobile monitor; and
   (c) processing the first signals received from the transmitter in real time using expert knowledge; and
   (d) providing one or more indications of results of the processing to the mobile unit.
   The results of the processing may include bio-feedback information of the user.
   The method may further comprise transmitting second signals from the mobile monitor to a remote server having an associated viewing station and providing an analysis of the second signals at the viewing station. The viewing station may include one or both of a remote call center and an interactive expert system.
   The processing may include calculating one or both of a parameter indicative of an arousal state and a parameter indicative of an emotional state of the user._Calculating a parameter indicative of an emotional state of the user may be based upon one or both of a sympathetic activity and parasympathetic activity of the user. Calculating a parameter indicative of an emotional state of the user may be based upon any one or more of an electro dermal activity, a heart rate, an electro dermal activity variability and a heart rate variability.
   The method of the invention may further comprise a step of displaying on a display associated with the mobile unit one or both of an image indicative of a parameter indicative of an arousal state of the user; and an image indicative of a parameter indicative of emotional state of the user. An image may include one or both of a two-dimensional vector and a color indicative of a parameter.
   The method of the invention may be used in obtaining respiration information selected from the group comprising duration of the inspiratory phase, and duration of the expiratory phase. The respiratory information m obtained from audio sounds produced during breathing or speaking. The respiratory information may be obtained by the user indicating the beginning of one or more inspiratory phases and the beginning of one or more expiratory phases of the user's breathing. A breathing rate of the user may be calculated based upon a heart rate variability of the user. The user's rate of breathing may be calculated based upon changes in an electrical skin capacitance of the user while the user is breathing.
   The method of the invention may further comprise training the user to increase any one or more of the followings: a duration of the inspiratory phase, a duration of the expiratory phase, and the ratio of the duration of the inspiratory phase to the duration of the expiratory phase.
   The method of the invention may further comprise displaying on a display associated with the mobile monitor an image indicative of bio-feedback information, wherein the image includes any one or more of the following: an image indicative of breathing activity, an image including a graph indicative of EDA activity, an image including a graph indicative of heart rate, an image including a graph indicative of heart rate variability and an image including a graph indicative of an autocorrelation of heart rate variability. The analysis of the second signals may include a recommendation for the user to improve a psycho physiological state of the user. The recommendation may be displayed on a display associated with the mobile unit.
   The method of the invention may comprise displaying a target value for one or more of the one or more obtained physiological parameters.
   The method of the invention may further comprise steps of:
   (a) challenging the user with one or more stimuli;
   (b) monitoring one or more reactions of the user to said one or more stimuli;
   (c) calculating, in a calculation based upon the one or more reactions, at least one parameter selected from the group of: latency time of a reaction, maximum reaction time, half recovery time, maximum stress, and new baseline stress; and
   (d) providing feedback to the user based on one or more of the calculated parameters.
   The method of the invention may be used in a method of self behaviour modification comprising any one or more of the methods selected from the group comprising:
   (a) cognitive behavioural therapy (CBT);
   (b) visualisation;
   (c) self hypnosis;
   (d) auto suggestion;
   (e) mindfulness;
   (f) meditation;
   (g) emotional intelligence skills;
   (h) psychological counseling provided over a communications network.
   When the method of the invention is used in a method of self behaviour modification the method may further comprise:
   (a) providing the user with an interactive introduction about a specific condition of the user;
   (b) providing the user interactive questionnaires for self assessment; and
   (c) providing the user with one or more interactive sessions selected from the group comprising:
   an interactive session for self training to implement cognitive techniques;
   interactive sessions for self training to implement behavioural therapy;
   interactive sessions for self hypnosis;
   interactive sessions for visualisation;
   interactive sessions for auto suggestions;
   interactive training to acquire and implement life and interpersonal relational skills;
   interactive training to improve emotional intelligence skills;
   interactive training to find purposes and goals; and interactive training to plan steps in life.
   The user may be provided with one or more interactive sessions while the user is in a deep relaxation state.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE DRAWINGS

An exemplary embodiment of the invention is described in the following section with respect to the drawings. The same reference numbers are used to designate the same or related features on different drawings. The drawings are generally not drawn to scale.

The invention is herein described, by way of example only. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

FIG. 1 is a physiology monitoring system, according to an exemplary embodiment of the invention;

FIG. 2 shows a sensor module attached to a user's finger, according to an exemplary embodiment of the invention;

FIG. 3 shows some details of a sensor module, according to an exemplary embodiment of the invention;

FIG. 4 is a schematic representation showing the mental and physiologic states of a person;

FIG. 5a shows a typical electro cardiogram (ECG) of a healthy person;

FIG. 5b shows a typical light reflection optical signal as affected by the blood flow;

FIG. 5c shows frequency analysis of heart monitoring signal;

FIG. 6a shows a graph of typical heart beat rate vs. time and its correlation to breathing cycle;

FIG. 6b shows frequency analysis of Heart Rate Variability (HRV);

FIG. 7a shows an exemplary display showing sensors output, according to an exemplary embodiment of the invention;

FIG. 7b shows an exemplary display showing heart beat rate (HR), according to an exemplary embodiment of the invention;

FIG. 7c shows an exemplary display showing Electro Dermal Activity (EDA), according to an exemplary embodiment of the invention;

FIG. 7d shows an exemplary display showing Heart Rate Variability, demonstrating the breathing cycle, according to an exemplary embodiment of the invention;

FIG. 8 shows an exemplary graph of stimuli induced stress used in a training session, according to an exemplary embodiment of the invention;

FIG. 9 schematically shows an electric circuitry of a reflective Photo-Plethysmograph with automatic continual adjustment of the source light intensity in accordance to an exemplary embodiment of the invention;

FIG. 10 shows an improved electronic circuit for EDA monitoring in accordance to an exemplary embodiment of the invention;

FIG. 11 shows an exemplary graph of the relationship between the user's skin resistively and voltage measured by improved electronic circuit for EDA in accordance to an embodiment of the invention; and

FIG. 12 shows an entertainment system according to an aspect of the invention.

### DETAILED DESCRIPTION OF EXEMPLARY EMBODIMENT

The following detailed description is the best presently contemplated modes of carrying out the present invention. This description is not to be taken in a limiting sense, but is made merely for the purpose of illustrating the general principles in accordance with the present invention. The scope of the present invention is best defined by the appended claims.

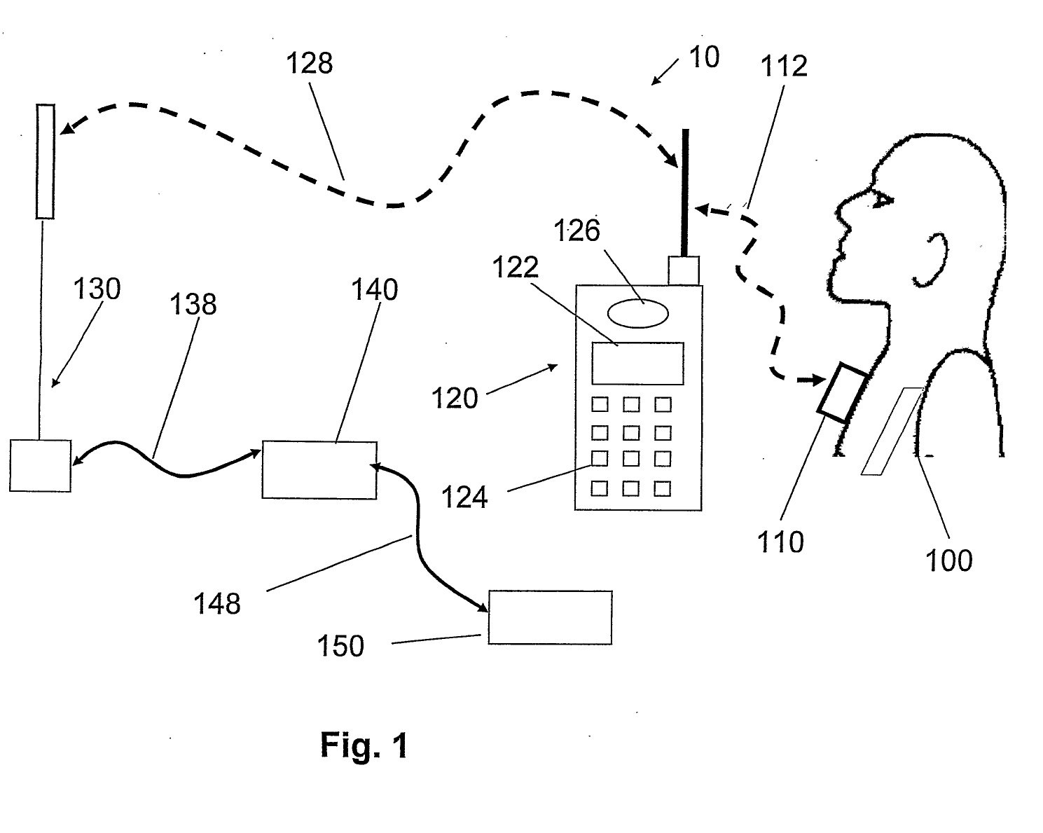

With reference to the drawings, in FIG. 1 shows a physiological monitoring system 10, in accordance with an exemplary embodiment of the invention.

A sensor module 110 is attached to a user 100. A communication link 112 is used to transfer data from the module 110 to a mobile monitor 120. Based on the transferred data the mobile monitor 120 provides visual biofeedback to the user by means of a display 122 and optionally an audio biofeedback to the user by means of speaker 126. Optionally, a keypad 124 is used to control the operation of the mobile monitor 120, sensor module 110, or both. Optionally the user can control the operation using voice recognition methods.

Optionally, a communication link 128 is used to connect the mobile monitor 120 to a remote server 140 where in-depth analysis of data obtained by the sensor unit 110 may be done and, optionally, data can be transmitted to an expert or another user. In the exemplary embodiment of FIG. 1, the mobile monitor 130 is a cellular phone, communication link 112 is a Bluetooth link, and communication link 128 is cellular RF link to a cellular base station 130 which is linked to a remote server 140 by a data link 138.

Optionally, an additional data link 148 such as Local Area Network (LAN) or Internet networking or RF cellular link connects the remote server 140 to a viewing station 150 where a human expert may provide interpretation of the data and transmit recommendations to the user.

Sensor Module

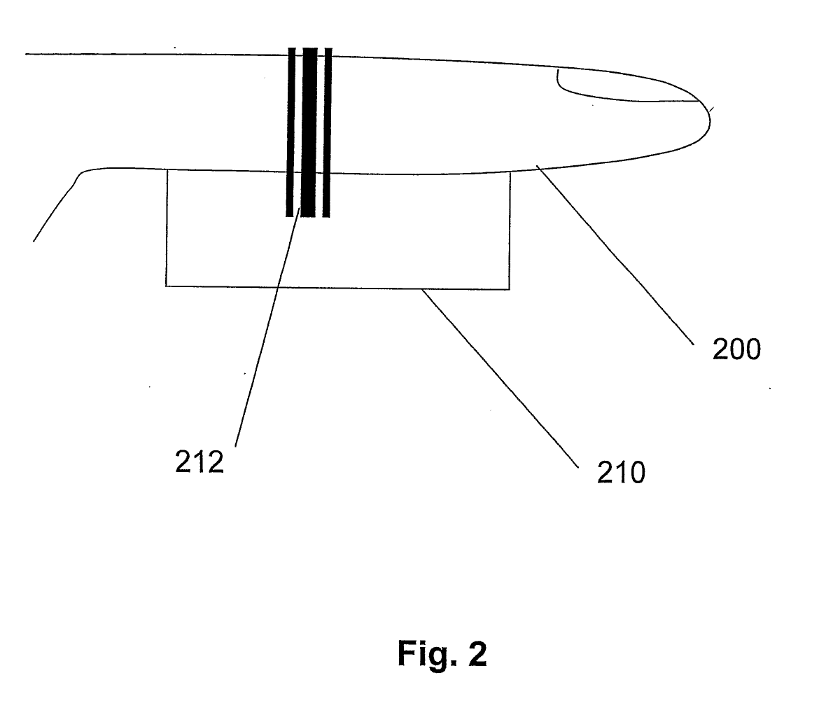

FIG. 2 depicts a sensor module 210 that may be used in the system 10 instead of the sensor module 110. The sensor module 210 is in contact with the user's finger 200. The sensor module 210 may be attached to the finger by a strap 212 as shown in FIG. 1, or the sensor module 210 may be shaped to fit over the finger. Alternatively, the finger 200 may simply be applied to the sensor module 210.

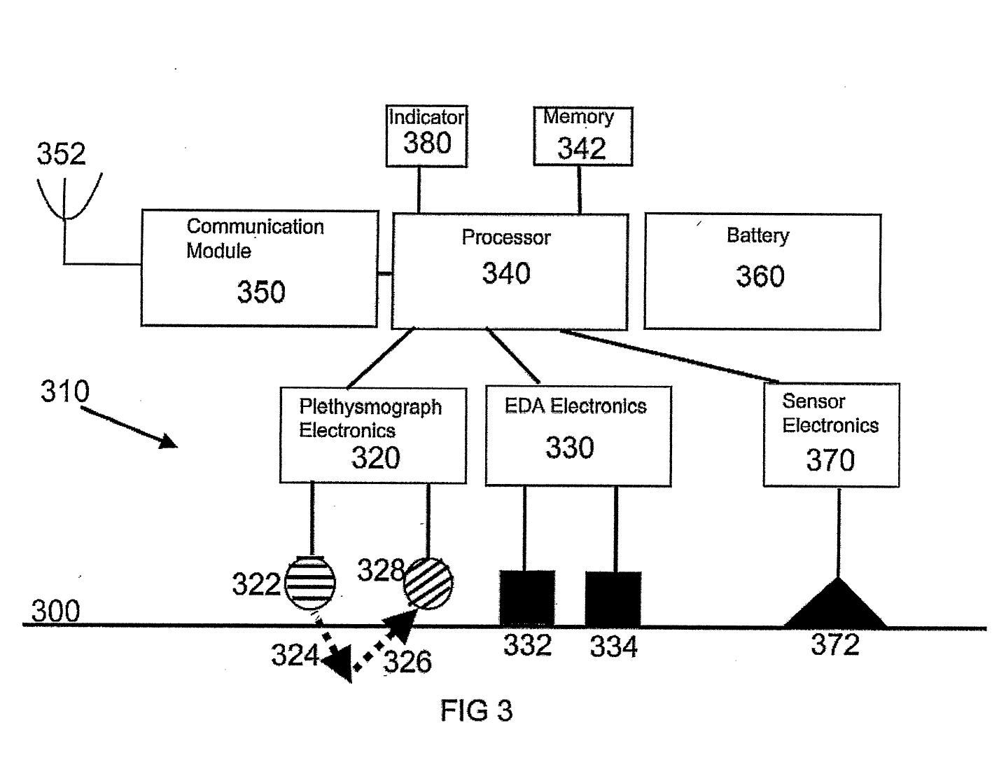

FIG. 3 shows a block diagram of a sensor module 310 for use in the system 10 according to an exemplary embodiment of the invention.

In the exemplary embodiment of FIG. 3, Electro Dermal Activity (EDA) at a user's skin surface 300 is monitored by applying at least first electrode 332 and second electrode 334 to the skin surface 300. EDA electronics 330 monitors the skin resistively by applying a very low electric voltage across the first and second electrodes and creating a minute electrical current between the electrodes. EDA electronics 330 generates a digital signal indicative of the skin resistively.

In the exemplary embodiment of FIG. 3, blood flow under the skin 300 is monitored by Plethysmograph Electronics 320 which is used for Heart Rate (HR) monitoring. In this exemplary embodiment, a light source 322 illuminates the skin surface 300 with emitted light 324. The intensity of scattered light 326 reflected from the skin and received by light detector 328 depends on the blood flow in the skin. Phethysmograph electronics 320 generates a digital signal indicative of the blood flow and thus may be used to monitor heart activity.

Optionally, one or more additional sensors 372 connected to additional sensor electronics 370 is used to monitor one or more additional physiological signals such as temperature, Electrocardiogram (ECG), blood pressure, etc.

The processor 340 receives digital data from EDA electronics 330, Phjethysmograph electronics 320 and optionally from additional sensor electronics 370 and processes the data according to instructions stored in a memory 342. The memory 342 may be a Read Only Memory (ROM) storing a pre-installed program, a Random Access Memory (RAM), a non-volatile memory such as flash memory or combination of these types of memory. The processor 340 may store raw or processed data in memory 342 for later use.

Optionally, the sensor module 310 is equipped with an indicator 380. Indicator 380 may provide visual or audio indication as to the status of the module such as “on/off”, “low battery”. Additionally or alternatively, indicator 380 may provide visual or audio indication as to the physiological state of the user based on the data from the sensors.

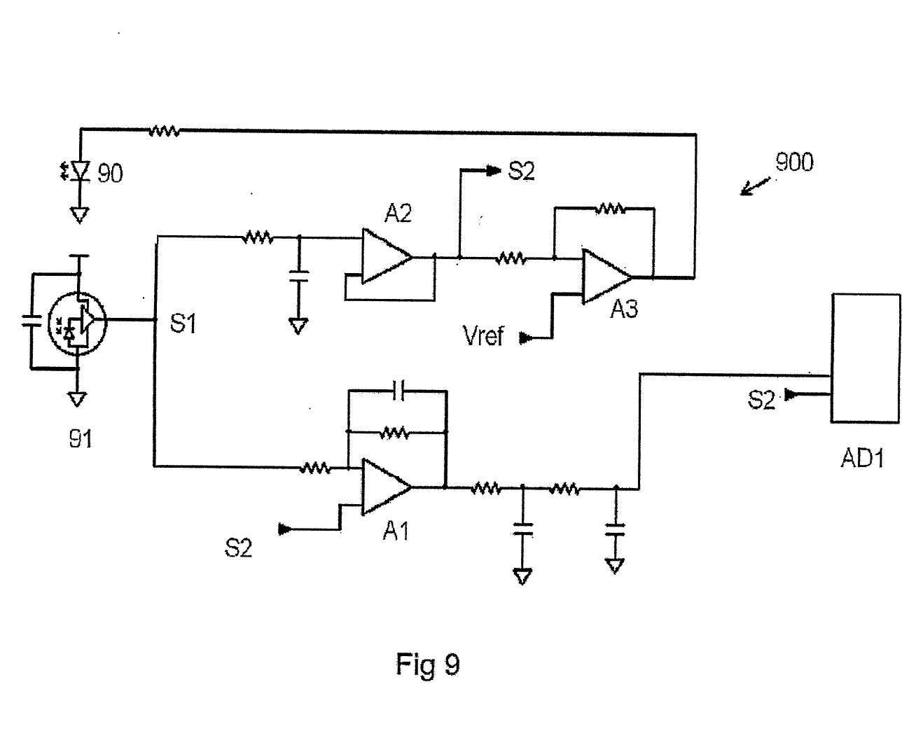

In the exemplary embodiment of FIG. 9, a communication module 350 is used as an interface between the sensor module 310 and mobile monitor 120 (FIG. 1). In this embodiment, a wireless communication link is used. Preferably, communication module 350 supports “blue-tooth” RF bidirectional wireless communication and is connected to antenna 352. Alternatively or additionally, Infra-Red (IR) communication, ultrasonic communication, WIFI communication, or wire communication may be used.

Battery 360 provides power for all the electronics within sensor module 310.

Alternatively or additionally, a wired connection, for example Universal Serial Bus (USB) may be used. In this case, a wired connection may provide power, optionally using electrical isolation such as a transformer which isolates the supplied power for safety, as well as means for data transfer.

The location of the sensor module on the user's body may depend on the type of physiological data to be acquired by the module and the type of sensor used.

For example, for measuring an EDA signal, the sensor's electrodes could be placed where the skin resistively changes depending on the person's stress or arousal level or any minute change in the autonomic nerves system, such as the palm of the hand, fingers wrist or ear lobe.

For measuring blood flow by optical reflectance, the module could be attached to locations where blood vessels are close to the surface such as the wrist, fingertips ear lobe etc, or the forehead to monitor blood flow in the brain.

For measuring cardiac electrical activity (ECG), the sensor may be attached to the user's chest using an adhesive or a strap, alternatively ECG can be monitored by attaching electrodes to two hands.

For temperature sensing, a sensor, which may be external to the sensor module, may be placed in the armpit or ear etc.

Alternatively, sensor may be temporarily touched to the measurement location for the duration of the measurement.

More than one sensor module may be used simultaneously. Two or more sensor modules may acquire the same or different physiological signals and communicate them to the same or different mobile monitors. Optionally, a plurality of sensors may monitor one or plurality of users simultaneously. The sensors may communicate with the same mobile monitor or with different monitors.

The communication link 112 is preferably bidirectional and continues while the sensor module is in operation. In such cases, the sensor module transmits information indicative of the user's physiological state to the mobile monitor for display and processing and receives commands and instructions from said mobile module. Such commands and instruction may control the operation mode of the sensor module. For example, the data sampling rate may be changed by such a command. Additionally or alternatively, data sampling accuracy or range may be changed by such commands. Programs executed by processor 340 may be uploaded and stored in the memory 342.

Alternatively, the communication link 112 may be unidirectional in which case the sensor module 310 only transmits information to mobile monitor 120. Optionally, the communication link 112 is intermittent. For example, for saving power and prolong battery life, the communication link may be activated only on demand, or when signals detected by the sensor are in specific ranges, for example: above or below thresholds or satisfy other conditions. For example, if the processor 340 detects an anomaly in the acquired physiological signal, it may initiate a data transfer to the mobile monitor. Alert conditions may be set up that trigger such data transfer to the mobile monitor 120. For example, heart rate may be monitored by the processor 340 to detect anomalous conditions regarding the rate and its Variability such as: Heart Rate (HR) too high, HR too low, Heart Rate Variability (HRV) too low. Breathing rate which may be inferred from analysis of HRV as will be demonstrated later, may also be used to trigger data transfer.

Alternatively or additionally, data transfer may be triggered by the mobile monitor.

For example, the mobile monitor 120 may be a laptop computer, The sensor module 310 may acquire and log physiological information, preferably in a compressed form in memory 342. Such a log may span a duration of several minutes or hours. When the sensor module 310 is in the vicinity of the mobile monitor, the acquired and stored data may be transferred on command initiated automatically or manually.

The data transfer rate may change depending on the operation mode of the sensor module. For example, one or few of HR, EDA ECG and HRV may be relayed to the mobile monitor during normal operation mode, while more or all of the signals are transferred during another mode of operation. Optionally, data is stored in a buffer, for example a cyclic buffer within memory 342 such that data recently acquired is available until over-written. Buffered data may be transferred on demand or initiated by the processor 340 or the mobile monitor.

Instructions and commands may be initiated by the remote server 140 or expert station 150 and relayed to sensor module 110 through mobile monitor 120. Alternatively, different communication methods may be used for different purposes. For example, data transfer from sensor module 112 to mobile monitor 120 may be achieved by a unidirectional communication such as IR transmission, while reprogramming the sensor module or setting alert parameters may be done while sensor 110 is connected to mobile monitor 120 using a USB cable. It should be apparent that other combinations of communication modes and methods are possible.

Preferably, the sensor module 310 comprises means for monitoring blood flow in the skin 300 using the phethysmograph electronics 320; light source 322 and light detector 328. In the preferred embodiment, the light source 322 is a Light Emitting Diode (LED) emitting red or IR light 324, or a plurality of LEDs transmitting same or plurality of wavelengths for example both red and IR light. Other light sources may be used such as solid-state diode lasers or Vertical Cavity Surface Emitting Laser (VCSEL). In the preferred embodiment, the light detector 328 is a Silicon photodiode. Optionally, the intensity of the emitted light 324 is not constant. For example, HR electronics 320 may turn off the light to conserve energy or to perform periodic calibration and ambient light subtraction. Additionally or alternatively, the intensity of emitted light 324 may be controlled by plethysmograph electronics 320 to compensate for different skin colors and person to person variations in skin light scattering properties such that reflected light 326 will remain within specific range. This method ensures that the light detector 328 and its associated amplifier and Analog to Digital Converter (ADC) will not be saturated or out of range. Alternatively the light source 322 may be placed on one side of the user's appendage such as finger or ear lobe and the light detector 328 placed on the other side of the appendage. In this case the detector detects the light transmitted through the appendage instead of the reflection light.

FIG. 9 shows some details of exemplary electric circuitry of a reflective photo-plethysmograph 900 with automatic continual adjustment of the source light intensity in accordance to an embodiment of the invention. The circuit is designed to pick up changes in light intensity as blood passes through the capillary bed of a user for example, in the finger. The intensity of reflected light intensity changes in time reflecting the pulsatile action of the heart in the user. The change is converted to a voltage, amplified, filtered and then digitized signal before being passed to a microcontroller 340

The interface sensor comprises an intensity-controlled Opto-transmitter Tx, preferably a red or Infra-red LED and a light receiver Rx preferably a photodiode or a phototransistor and a trans-impedance (current to voltage) amplifier. In the preferred embodiment, the receiver Rx is an integrated component including both photo-detector and amplifier. The signal S1 from the output of the trans-impedance amplifier is feed to one input of a differential amplifier A1 and is also low-pass filtered and taken to a unity-gain buffer amplifier A2 giving output signal S2. Output signal S2 represents the average level of light falling on the opto-sensor with any pulsatile component removed due to the low pass action of the filter. S2 is then used as the other input to the differential amplifier A1. The output from A1 is low-pass filtered and then fed along with S2 to the differential inputs of an analogue to digital converter AD1 providing a digitized pulse signal to microcontroller 340. Additionally, S2 is used along with a fixed reference voltage Vref slugged comparator A3 whose output controls the intensity of the opto-transmitter Tx. This allows optimal biased input conditions for the receiver by automatic continual adjustment of the source light intensity. The overall effect of the circuit provides for wide variability in ambient light conditions, skin tone of the subject and minimizes unnecessary current drain due to optimal control of the light source. It is possible to replace the photo-plethysmograph with a piezo-electric sensor, which monitors minute changes in blood vessel pressure instead of changes in reflected light.

Another aspect of the invention is a GSR EDA sensor. GSR and EDA have been used for many years to monitor general arousal levels. However, efficacy has been compromised because the difference between skin resistance/impedance of individuals is very high as is the disparity encountered within the same individual experiencing differing emotional and physiological states.

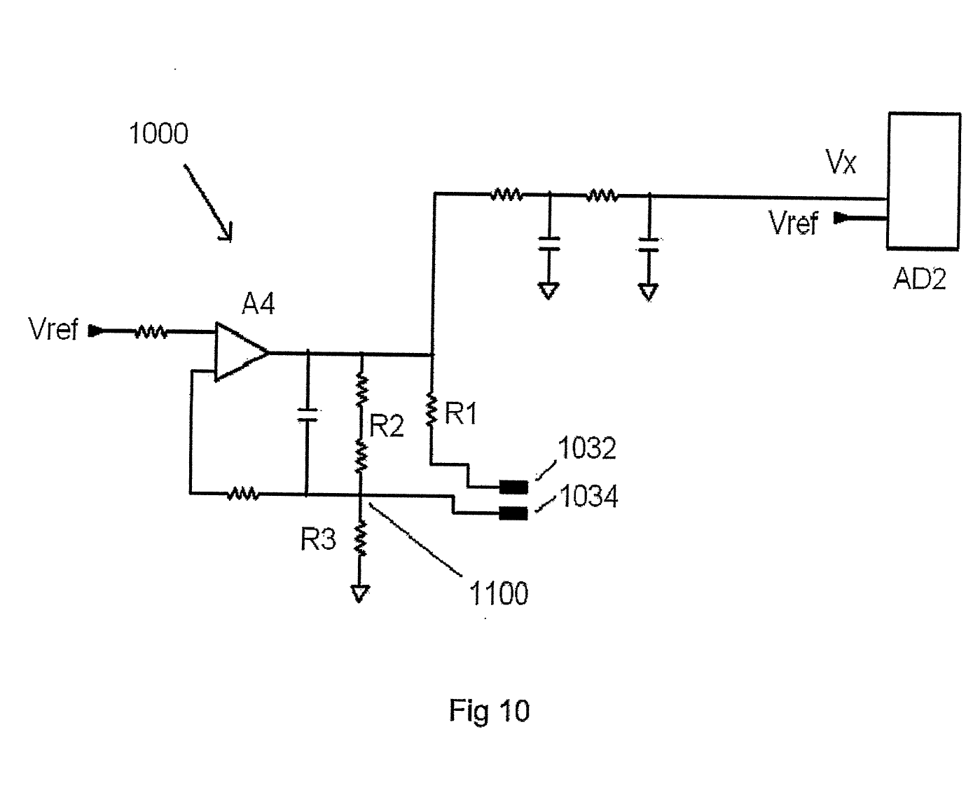

In order to accommodate a wide spectrum of users, current systems are not sensitive enough to diagnose minute changes. One way, used in the art, to overcome this problem is to have two reading sessions monitored by experts: the first reading creates a base line, the second session is done at higher sensitivity centered around the baseline. In the present invention,
A 16-bit Analog to Digital Converter (ADC) microchip is preferably used to cover a larger range with high sensitivity.
The electronic circuit, as depicted in FIG. 10, is modified to enhance dynamic range.
Software is used that can automatically monitor both the user's base line and level of sensitivity, and display it to the user in an understandable way.

In contrast to prior art EDA units which use 8-bit or 12 bit ADC, the EDA electronics 330 of the preferred embodiment uses a 16-bit ADC. It was discovered that small temporal changes in skin resistance provide significant physiological information, while the EDA may change over a wide range. Additionally, the large dynamic range reduces or eliminates the need to manually adjust the ADC range or baseline or sensitivity. Since the EDA signal is low bandwidth, high accuracy ADC such as “sigma delta” type may be used.

Optionally, automatic auto ranging and auto scaling may be used. In this method, a baseline may be subtracted from each measurement. The subtracted value may be stored or transmitted to the mobile monitor so that actual values may be restored. Similarly, automatic scaling may be used to re-define the signal change associated with each bit of the ADC. Optionally or additionally, a Logarithmic or other non-linear scaling of acquired data may be used.

FIG. 10 shows an electronic circuit 1000, for using non-linear scaling for EDA monitoring. The circuit 1000 is designed to pick up very small changes in sweat gland activity reflecting changes in the emotional arousal of the user. The circuit monitors changes in skin resistance level, which are then amplified, filtered and digitized before being passed to the microcontroller 340.

In one preferred embodiment, the interface consists of a pair of gold plated finger electrodes 1032 and 1034, etched onto a PCB. The EDA signal has a large dynamic range and there are also very large variations between subjects of base skin resistance level. The electronics comprise a modified constant current source. Operational amplifier A4 tries to maintain the potential at intersection 1100 at voltage Vref, providing a fixed current through the resistor R3. This current is the current flowing through the combination of resistor R1 and the EDA electrodes 1023 and 1034. The voltage Vx, required to maintain this constant current is measured with respect to reference voltage Vref and digitized by Analog to Digital Converter AD2 after low-pass filtering. Preferably, AD2 is a 16-bit ADC.

The resistance R2 is preferably high, for example (R2>10 times the normal subject base readings) and during normal operation has no significant effect i circuit. However for subjects with high levels of basal skin resistance, R2 becomes more significant and the voltage output from A4 is reduced to prevent output saturation. This allows subjects with high base resistance to be measured using the same circuitry with the output measured with a non-constant current.

The measured voltage Vx is given by:

$$Vx = \frac{Vref / R 3}{1 / ( R 1 + Rx ) + 1 / R 2}$$

where R1, R2 and R3 are the resistor values. Vref is a reference voltage value, and Rx is the changing resistance of the user's skin appearing between the electrodes.

An EDA monitoring device based on the circuitry according to the current invention may be capable of measuring small changes in the skin resistance over a large range, for example from 50 KOhms (50,000 Ohm) to 12 MOhms (12,000,000 Ohm). The exact range may be adjusted by changing the values of the components in said circuitry.

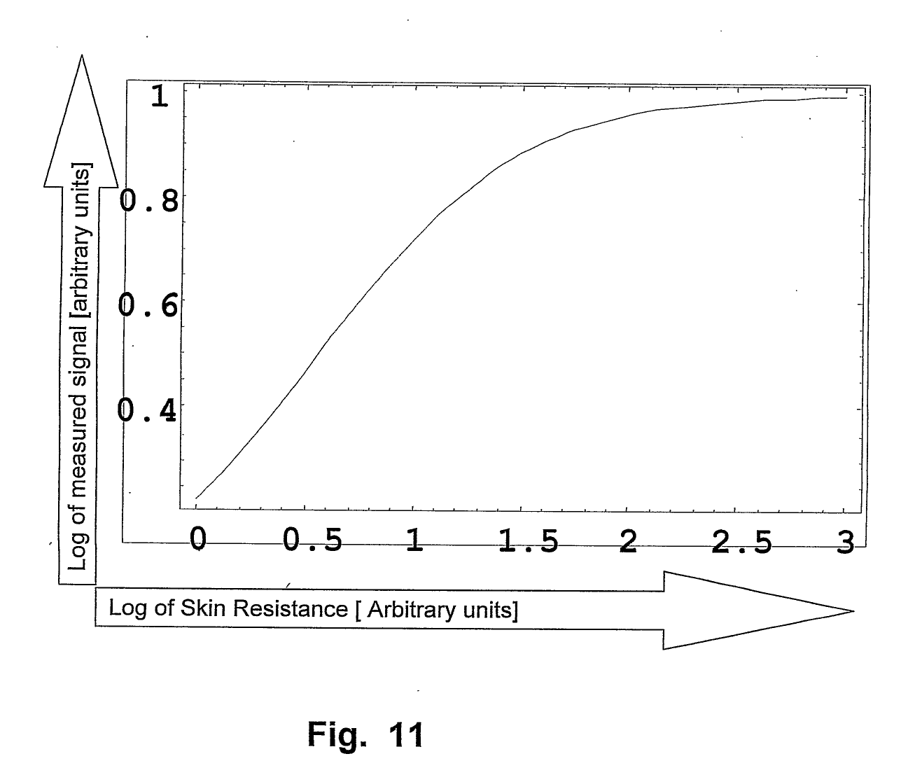

FIG. 11 shows an exemplary graph of the relationship between the measured voltage Vx and the user's skin resistively Rx plotted in arbitrary units on a log-log scale. A linear range is observed near the origin. The plot becomes non-linear for high Rx values.

Optionally, the sensor module 310 is equipped with an indicator 380. Indicator 380 may provide visual or audio indication as to the status of the module or provide one or few of: visual, vibrational, or audio indication as to the physiological state of the user based on the data from the sensors. For example, indicator 380 may be used to alert the user that a physiological signal is out of the predefined range. The alert may be initiated locally by processor 340 or communicated to the sensor module through communication link 112. Optionally, indicator 380 may be used as biofeedback in a training session as will be detailed later.

The indicator 380 may comprise an LED or a few LEDs optionally of different colors. Optionally, indicator 380 may comprise a speaker providing audio signal to the user. Optionally, indicator 380 may comprise a means to produce vibration such as PZT buzzer or miniature electric motor so that the alert may be sensed by the user and no one else.

Mobile Monitor

In an embodiment of the current invention the sensor module 110 is connected by communication link 112 to a mobile monitor 120. In one preferred embodiment, the mobile monitor 120 is a cellular phone or a Personal Digital Assistant (PDA) equipped with a processor to perform data analysis, memory, a display, Audio output, input means such as keypad, and microphone and sketchpad and means to communicate with both sensor module and remote server.

Specific programs necessary for interfacing with the sensor module and for providing feedback to the user may be uploaded by the user. For example, the program may be loaded into a cellular phone wirelessly in the same way a new game or ring tone is loaded.

Alternatively, other personal computing devices may be used as mobile monitors, for example a Laptop Personal Computer LPT or a media player such as Apple iPOD®, pocket PC or an electronic note-book. Alternatively, a standard PC may be used if the user wants to execute a training session without moving around or if the user wants to download data stored in the sensor module periodically or to reprogram the sensor.

The communication range of the sensor module is limited due to its small size and low battery capacity to few meters or up to almost 100 meters using Bluetooth. In contrast, the Mobile monitor is equipped with means to connect to a remote server wirelessly over the cell network, preferably using the Internet. For example, cellular phone may be connected using one of the cellular data exchange protocols such as GPRS. Other standard and proprietary protocols may be used such a wired connection to a phone line using a modem or an Asymmetric Digital Subscriber Line (ADSL), a Local Area Network (LAN) Wireless LAN (WAN), etc.

Remote servers may provide additional processing of the sensor's data, initial and updating of mobile monitor and sensor module programming, feedback and recommendations to the user, issue alerts to the user or summon rescue teams to a the user in emergency. Some mobile monitors may be equipped with means to establish their physical location such as Global Positioning System (GPS) which may be used to direct the rescue team to a user in distress such as during cardiac mishap or epilepsy episode.

States of Mind

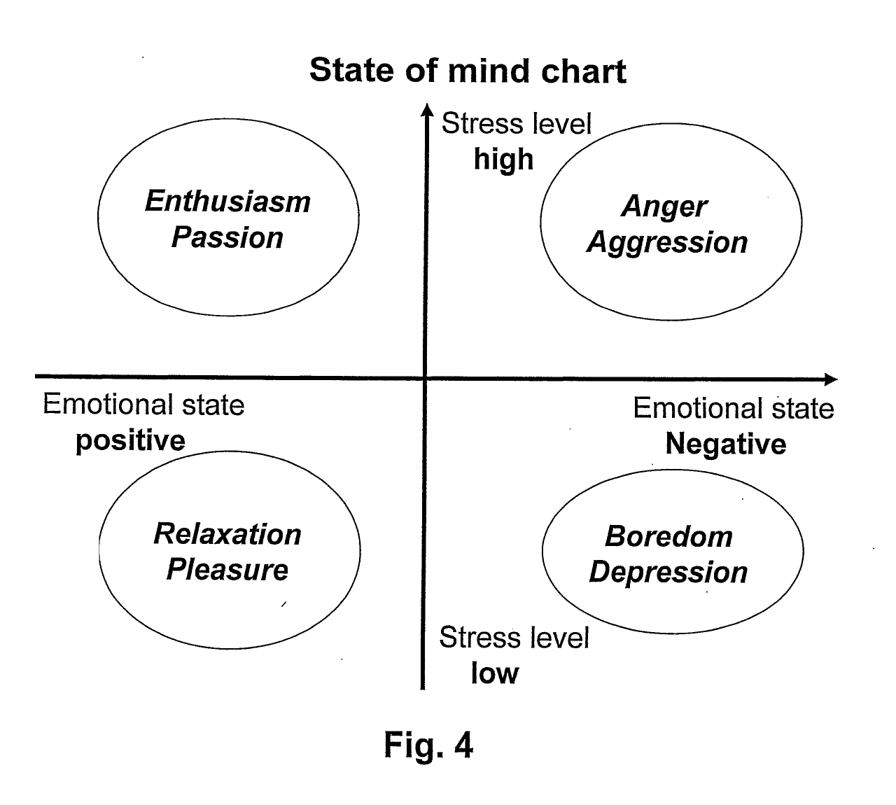

Reference is now made to FIG. 4 illustrating schematically examples of possible “states of mind” of a user. The vertical axis is the arousal level of the user while the horizontal axis is his emotional state.

Biofeedback and monitoring systems are not designed to analyze emotions. The GSR or EDA sensor reflects arousal level, but the system cannot differentiate between positive arousal—that is when the user is enthusiastic and negative arousal when the user is stressed and angry. The existing methods also cannot differentiate between positive low arousal—when the user is relaxed and meditating, and negative low arousals—when the user is depressed and despondent.

Reference is now made to FIG. 4 illustrating schematically examples of possible “states of mind” of a user. The vertical axis is the arousal level of the user while the horizontal axis is his emotional state.

By integrating a sensitive EDA sensor (such as disclosed herein according to the current invention), HRV analysis, and optionally a multimedia display (such as a smart phone, PDA or PC), it is possible not only to analyze the state of the emotions of the user as described in FIG. 4, but also to train the user to improve his state of emotions and physiology.

For example: the system can have several modes of operation:

a) Baseline calibration: The system automatically determines a base line of the specific user. The base line includes vectors of parameters which will be calculated and recorded during the first interval, including: minimum, maximum and average HR, HRV, FFT (Fast Fourier transform), respiration rate (which can be calculated indirectly or monitored directly), and EDA—max, min, average, variance, number of fluctuation and slope.

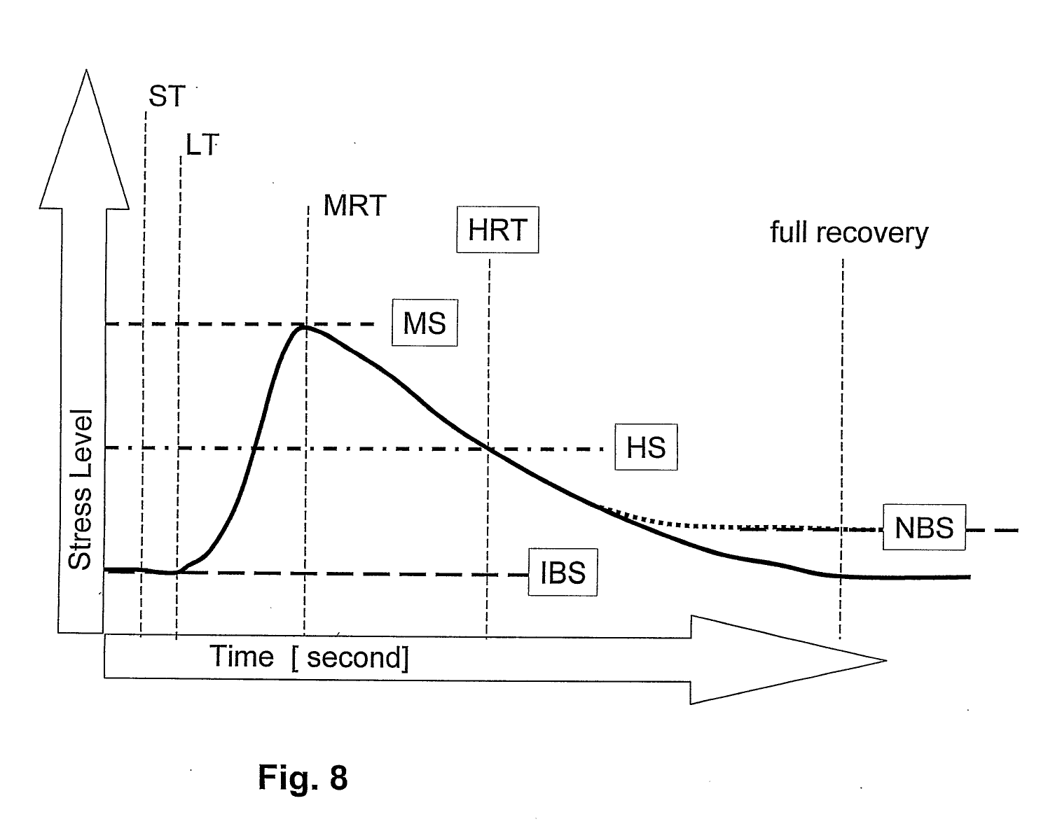

b) Calibration using induced state of mind: Preferably, a short time after the base line has been stabilized, the system presents prerecorded triggers. Each trigger is designed to elicit specific emotions in the user. The triggers may be prerecorded scenarios which can cause specific emotional reactions. The preferred methods are multimedia methods, which can be a prerecorded audio visual movie on a smart phone or PC. For a professional system, this can be virtual reality goggles with a real 3D scenario. For a less expensive system, the trigger can be only an audio session using a mobile phone. These triggers or scenarios can be general scenarios which have been tested and validated in the past to create a specific emotional reaction, or can be customized for a specific culture, people or person. For example, a scenario might be an audio visual display of a dentist drill in a tooth, or a car accident for negative arousal; winning a game, or a romantic relationship for a positive arousal; a relaxing nature movie for positive relaxation, and a boring and sad scenario for negative low arousal. Before, during and after each trigger, the system monitors, calculates and records the vectors of parameters as described above and calculates the parameters which are described in FIG. 8 when each trigger start and finish.

c) Calibration using user reported state of mind: The system can ask the users to input their subjective feeling, for example, by using the keyboard of their cell phone (e.g.) if you feel very happy press 9, very sad press 1). By calculating the above vectors and correlating them with the specific triggers, the system is able to differentiate between specific states of emotions and to correlate them with the physiological state of the user. The system can keep those vectors and their correlations to specific emotional states for specific users, and/or for each group of users.

d) Learning mode: The system can incorporate neural network and similar methods to continue learning, using the data from a group of users in the past to predict the emotional state of a specific user in a shorter time using his vector of data as describe above. For example, using this algorithm with a group of people, the system can predict that when a user has a low HRV and at the same time a high skin conductivity, his emotional state is “negative stress”, while user with a high HRV and a low skin conductivity is “relaxed and positive”.

e) Training mode: The system can also train the users first to be more aware of their physiological and emotional state during their daily activities, and, second to acquire better behavioral, physiological and psycho-physiological habits, such as increasing their respiration cycle, and the ratio of expiration to inspiration, increasing their HRV, and learning to relax. Third, the system can be used to train users to improve their reaction and responses to negative triggers and events during their daily life, and to improve their reactions and performance under pressure. The system can simulate real events and train the user to improve his reaction, performance and behavior. For example while prior art biofeedback systems can be used only in an artificial setting (e.g. the therapist's office) the wireless sensors of the invention can be used during actual important activities, such as driving, playing music, competing in sports, during exams, work interviews, etc.

The system of the invention may be calibrated or customized to a specific user. Alternatively, statistical parameters acquired by studying the general population or a specific sub-group of the population may be used. In some embodiments, a remote server receives data from a plurality of users, optionally including information about the user, and uses the information to create a data set used for state of mind analysis. Optionally, parameters extracted from the data set are transmitted to the mobile units of at least some of the users to be used for determination of the state of mind of the users. Optionally, a study group or plurality of study groups of users are used by the service provider in order to create the data set. This real time analysis of the state of mind of the users including their emotional reactions to specific triggers can be used to train the users to improve their performance and also to analyze their reactions to specific events, triggers, products and services.

The users can receive feedback in real time directly from the system by audio-visual feedback in real time, and at the same time the system can transmit the information to an expert or coach who can help them improve their reactions. This can be relevant for health issues—e.g. a child with asthma who can get feedback in real time from the system and/or physician, or an athlete receiving feedback to improve his performance. For training and analysis, it is recommended to record the physiological vectors as described above together with the external situation—e.g. a video of the competition, or a musical performance. In this way, it is possible to find the correlation between the best performance and the physiological vectors, and to train the user to optimize his physiological, emotional and mental performance, using simulation of the event by video or visualization together with the real time feedback of the sensors.

Schematically, the upper section of FIG. 4 is characterized by high arousal state, such as physical or emotional stress. This stress may be a result of vigorous physical activity or by emotional state of anger, aggressiveness, fear, or anxiety. Alternatively, high arousal may be a result of excitement caused by constructive thoughts such as concentrating on performing a task, or feelings of enthusiasm or passion. These two different states are separated by their being on the right (negative emotionally) and left (positive emotionally) sides of the figure respectively.

Similarly, low stress states of mind, schematically symbolized by the lower half of the figure, may be a result of depression or boredom, characterized by low arousal or energy level and negative emotions on the lower right of the figure; or relaxation and self contained pleasure on the lower left side of the figure.

In an embodiment of the invention, the combination of sensors and data processing enable automatic determination of the state of mind of the user and may be used to provide feedback and interactive multimedia training to achieve and maintain the positive state of mind and body.

A high stress state is characterized by a high production of adrenalin hormone associated with high HR. However, high HR by itself cannot separate enthusiasm and passion from anger and anxiety. Positive mental states (left two quadrants of FIG. 4) are associated with secretion of growth hormone and dehydroepiandrosterone (DHEA), and characterized by high heart rate variability (HRV) and high skin resistance. In contrast, negative mental states (right two quadrants of FIG. 4) are associated with secretion of Cortisol hormone and characterized by HRV. Additionally, a state of relaxation is characterized by slow, steady breathing with slow exhale periods.

In an exemplary embodiment of the invention the state of mind is characterized by a two component vector: Emotional level: Left—more positive emotions, right-more negative emotions—on the horizontal axis; and Stress level on the vertical axis: Up—more stress, down—less stress.

In some embodiments of the invention a marker, for example an icon is displayed in the coordinates representing the state of mind vector and may be viewed by the user to allow monitoring of his state. The location of the marker may be periodically updated as the state of mind changes.

Alternatively or additionally, color codes may be used to symbolize the state of mind. For example, the horizontal axis may be represented by shades of yellow on the left to black on the right; while the vertical axis may be represented by shades of red on the top and blue on the bottom.

The combinations of these colors yields: Orange—representing a passionate mood on the upper left quadrant of the two dimensional scale; Green—representing a relaxed mood on the lower left quadrant; Dark Red—representing an aggressive mood on the upper right quadrant; and Dark Blue—representing depression on the lower left quadrant.

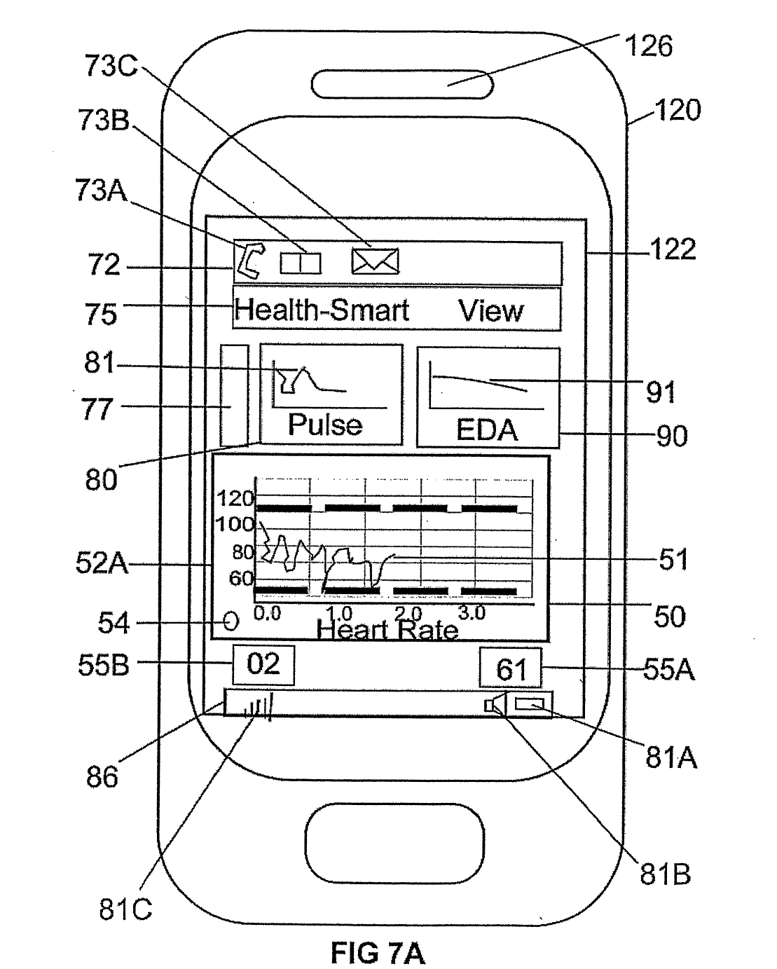

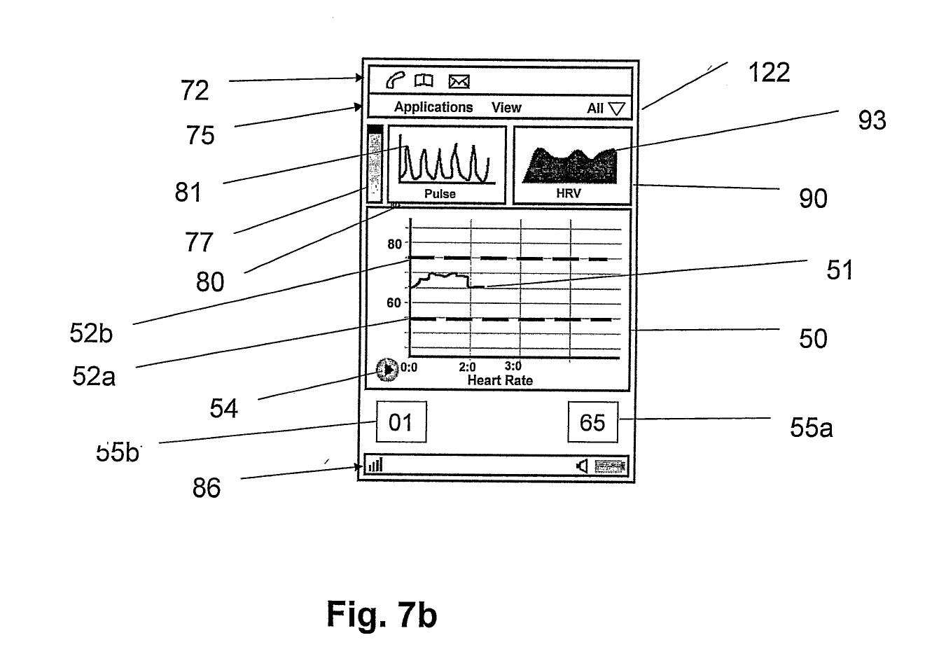

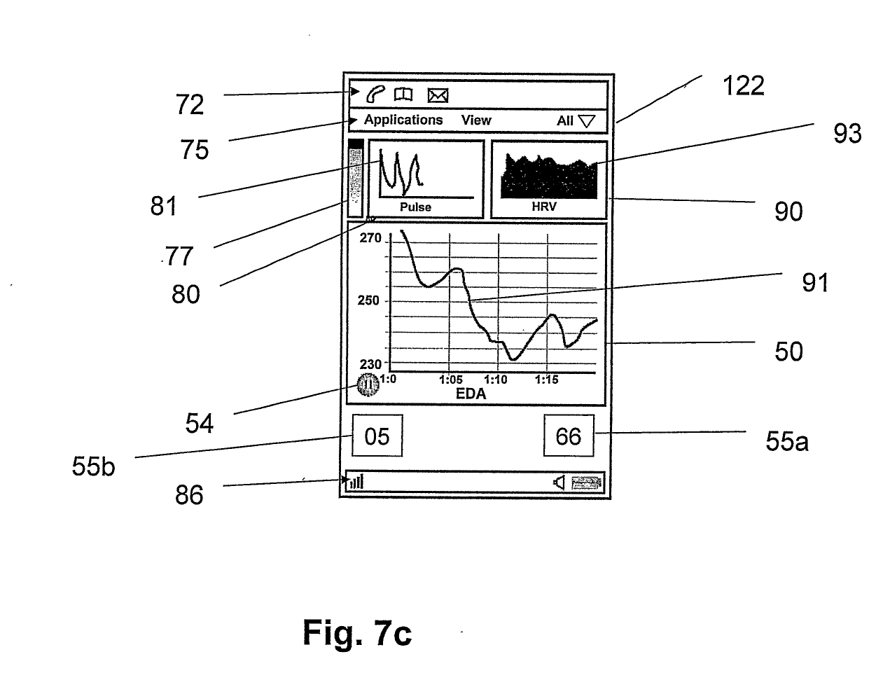

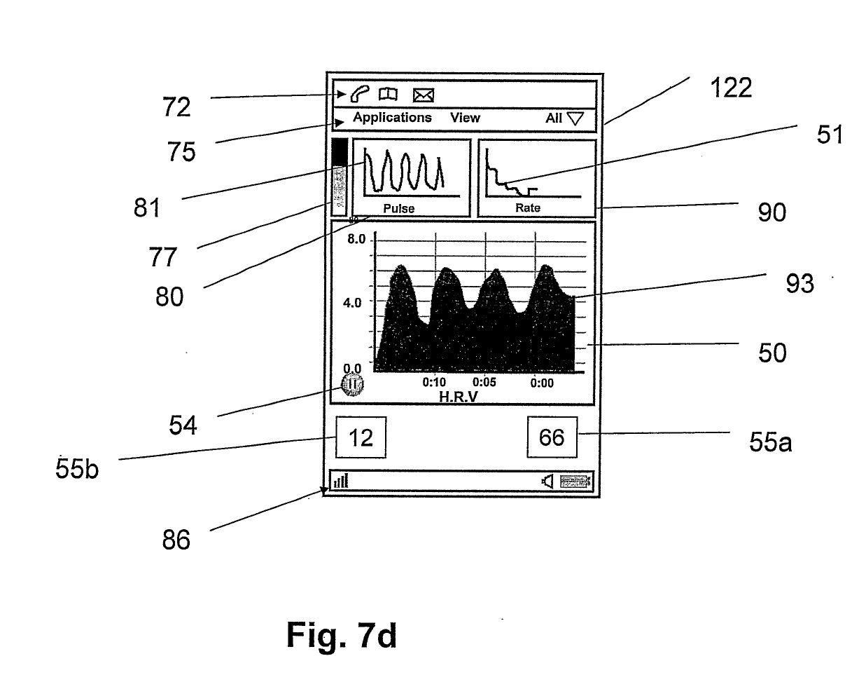

The resulting combination color, representative of the state of mind may be displayed on the display 122 of unit 120. For example, the resulting combination color may be used as background for one or some of the graphs as depicted in FIGS. 7a to 7d. It should be clear that other color schemes may be used within the general embodiment of the current invention. Such a color representation of state of mind is easy to view and may be intuitively understood by the user without the need to carefully observe the monitor or while performing other mental or physical tasks.

Data Processing

In an embodiment of the invention, heart pulses are tracked by data analysis performed by processor 340 within the sensor module.

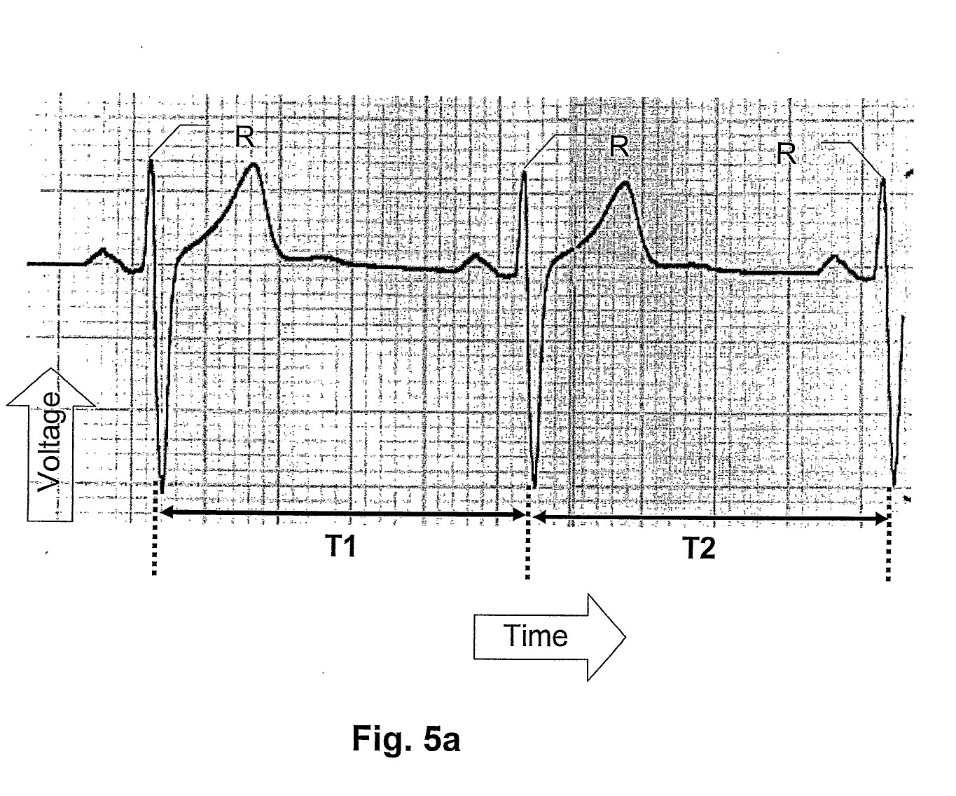

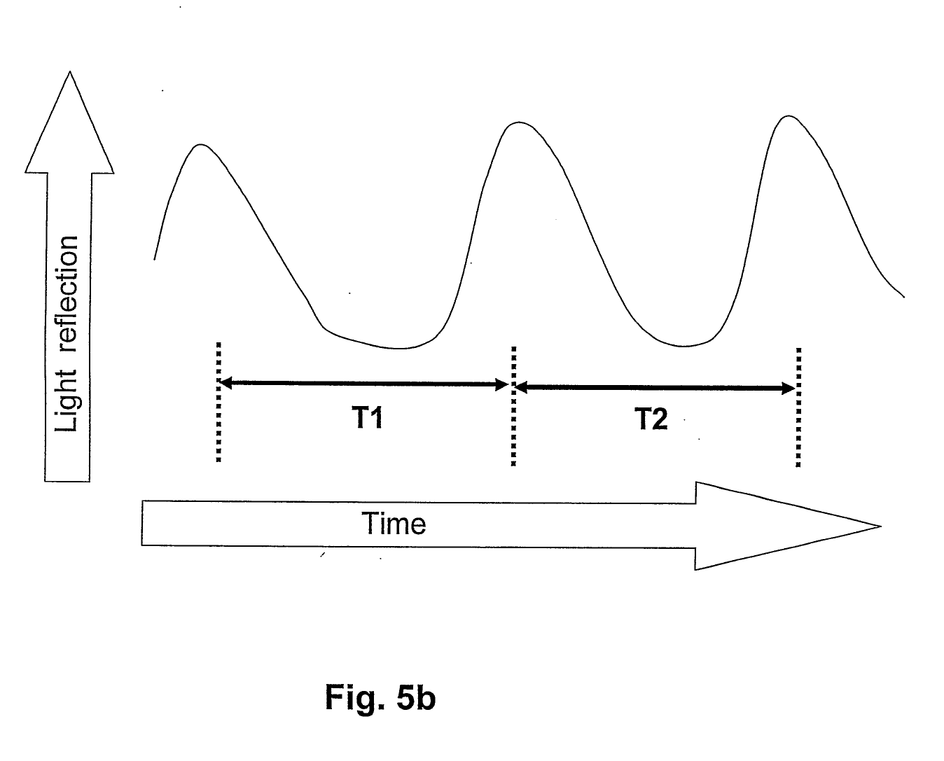

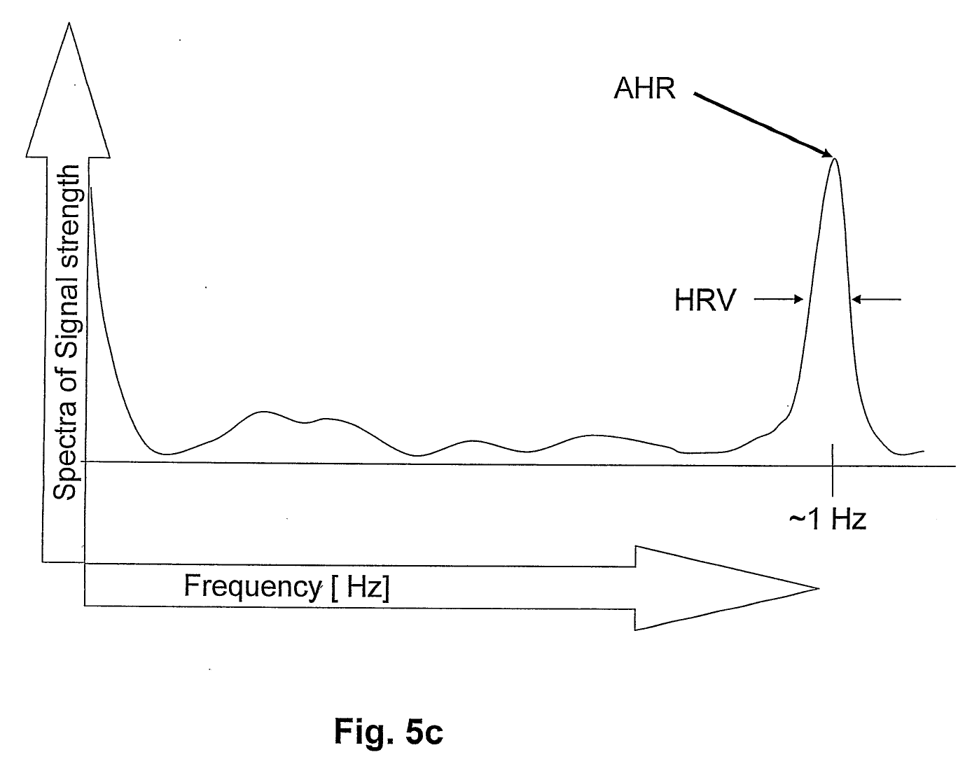

FIG. 5a shows a typical ECG signal of a healthy person. Three heartbeats are clearly seen separated by time intervals T1 and T2.

FIG. 5b shows a typical optical signal. Three heartbeats are clearly seen separated by time intervals T1 and T2.

In an embodiment of the invention, optical signals from detector 328 are analyzed and individual heartbeats are determined.

This can be done by identifying the peaks, minima or zero crossings in the signals, by performing auto correlation or by wavelet analysis.

In one preferred embodiment, local maxima are found in the optical signal. Then, the system checks if this peak is a heartbeat peak or only a local maximum due to noise. This determination may be assisted by performing comparison with signals from previous heartbeats and using, for example, probabilistic, heuristic or fuzzy logic algorithms.

In contrast to standard heart rate monitors which display only an average heart rate, the combination of the electronics and the peak detector—heartbeat recognizer algorithm enables the system to detect, calculate and present more accurately each heartbeat.

A similar analysis may be performed on an ECG signal if available. It is easier to detect accurate peaks in an ECG because the R wave has high amplitude and is sharp. The instantaneous HR is define as HR(t)=1/_{Ti}, where T(i) is the duration of heart cycle “i” (T is also known as R-R duration as seen in FIG. 5a), HR(t) is tracked over time (t) and optionally stored in the memory 342. Alternatively, the T(i)'s may be stored.

An average HR (AHR) may be calculated by averaging the values of HR over a specific period. A running average may be calculated over a predetermined time window to reduce noise in the signal.

HR Variability (HRV) may be calculated by several methods. One of them is the absolute value of the difference between the AHR and HR(t), and calculating the average of the HR(t) in the specific interval.

Other methods are calculation of the standard deviation or variance of the HR in a specific interval.

Optionally or additionally, spectral analysis of a heart signal may be performed. A computational efficient Fast Fourier Transform (FFT) algorithm is preferably performed to calculate the spectrum.

FIG. 5c shows a typical Fourier spectrum of heart signal. AHR can be inferred from the location of a peak, that is typically located between 0.5 to 3 Hz corresponding to an average heart rate of 30 to 180 beats per minutes. HRV may be inferred from the width of the peak.

A stress level can be inferred from the AHR wherein a high level of stress is characterized by higher than normal AHR. It should be emphasized that “normal” AHR is different for each individual and depends on age and physical stamina. Thus, this level may need to be updated from time to time, for example by measuring and averaging the AHR over an extended duration or by measuring it during a calibration session while the person is in a known state of mind. Similarly, the two ends of each axis may be calibrated during training and calibration sessions, for example: vigorous physical exercises vs. meditation rest or sleep.

Variability in HRV may be assessed from width of the peak in FIG. 5c.

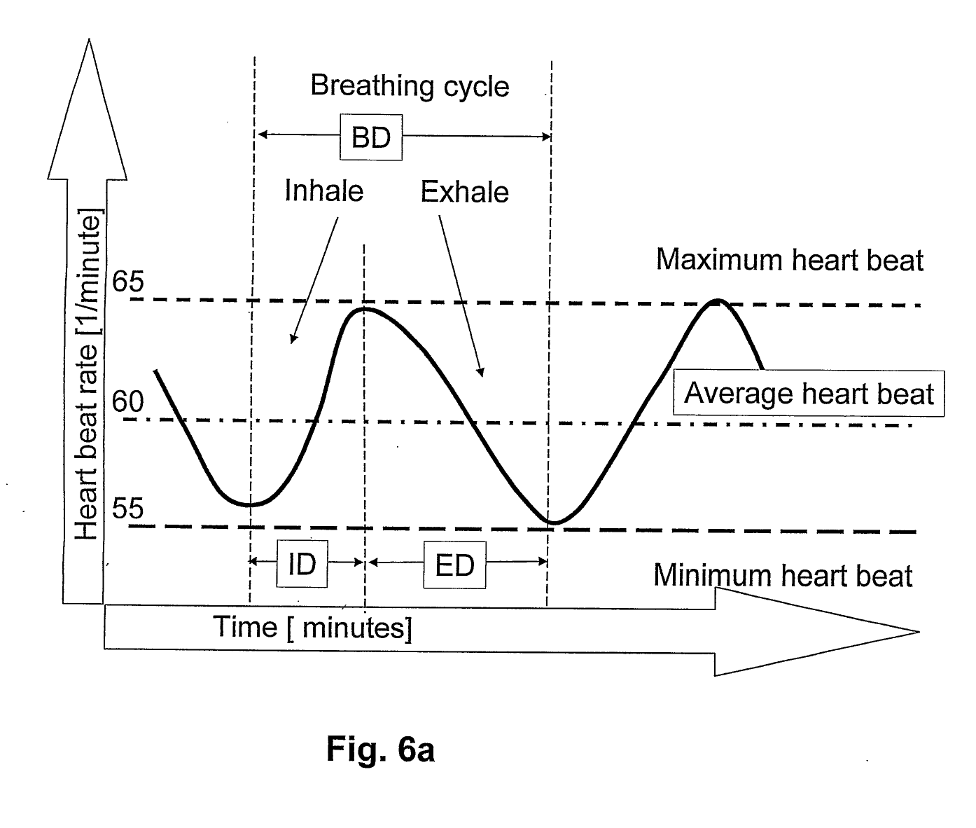

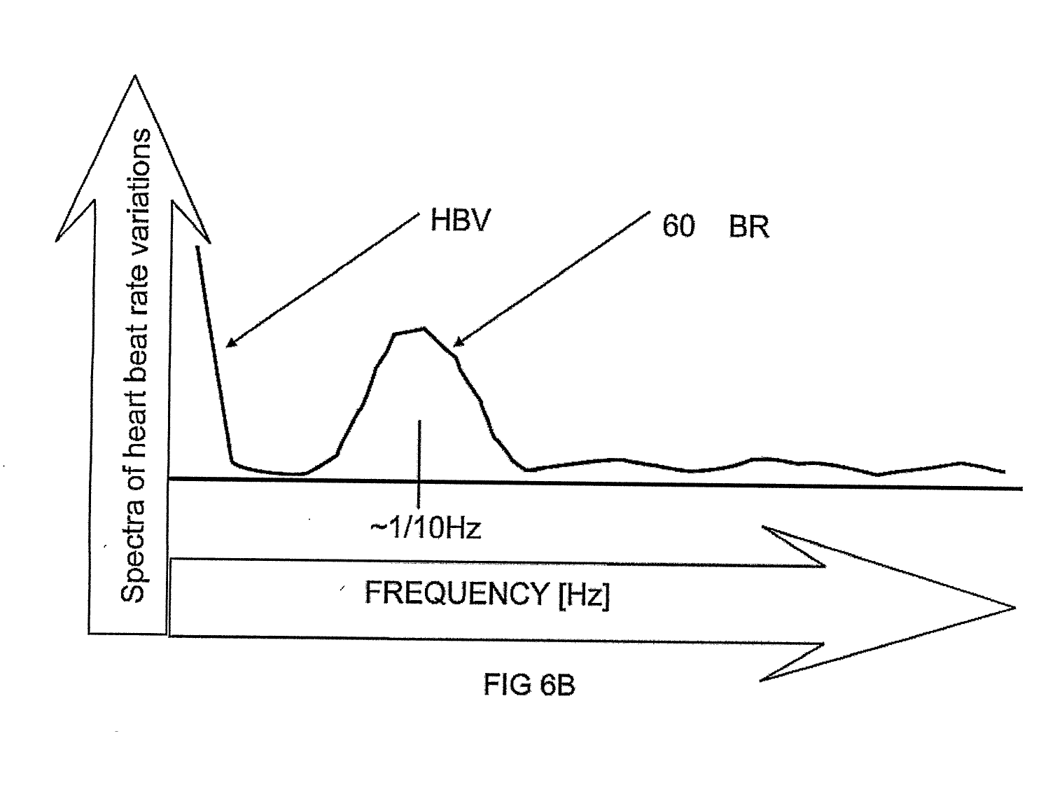

It was discovered that heart rate is correlated with the breathing cycle and autonomic nervous system functionality. FIG. 6a shows a typical graph of a healthy person's HR as a function of time during normal breathing cycle. The HR increases during inhalation and decreases during air exhalation.

Breathing monitors known in the art use strain gauge sensors strapped around the chest, or air movement sensors positioned near the person's mouth and nostrils. Using these sensors is cumbersome and uncomfortable. In contrast, an embodiment of the current invention infers the breathing from HR information.

In an embodiment of the invention, values of instantaneous HR(t) determined for example from optical signals or from ECG signals are analyzed and the breathing cycles are determined. This can be done by identifying the peaks, the valleys or zero crossings in the HR sequence, by performing auto correlation or using FFT analysis or by wavelet analysis. Each breathing cycle may be analyzed for Breathing Rate (BR), Breathing Depth (BD) and the Ratio of Exhale over Inhale duration (REI). Alternatively or additionally it can be analyzed and presented as two parameters: Inhalation duration and exhalation duration (average duration in seconds).

Where: BR per minute is defined as 60 over the duration of the breathing cycle in seconds;

BD is defined as the Minimum HR subtracted from the maximum HR during the breathing cycle normalized by the AHR, and

REI is defined as exhale duration divided by inhalation duration.

These values may be transmitted to the mobile monitor and optionally stored in the memory 342. Alternatively, the breathing analysis may be done at the mobile monitor.

The average values of BR, BD and REI (ABR, BD and REI respectively) may be calculated by averaging the values of BR, BD and REI over a specific period. A running average may be calculated over a time window to reduce noise in the signal.

Optionally or additionally, a spectral analysis of HR or HRV sequence, using a computational efficient Fast Fourier Transform (FFT) algorithm, is performed to calculate the spectrum.

In some embodiments of the invention, HR(t) is displayed to the user, for example as shown in FIG. 7a. A graph of HR(t) may be useful for assessing the ability of the user to quickly adapt to changing circumstances, for example to regain a calm mood after an exciting stimulus.

An additional method to analyze the data and extract breathing pattern is to perform autocorrelation on the HR(t). Autocorrelation, AC(k) may be defined as the sum over a specific interval j={t-K to t} of HR(j)*HR(j-k). In some embodiments of the invention, the autocorrelation function is displayed to the user to assist visualization of the breathing cycle as will be seen in FIG. 7d. When breathing is steady, the autocorrelation function exhibits a deep wave pattern with a cycle's length equal to the breathing rate. The depth of the waves of the autocorrelation function is indicative to the depth of the berating. In contrast, when the user is in agitated state of mind, the breathing is unsteady and may be shallow, causing the autocorrelation function to flatten. The autocorrelation function may be used for calculating the Breathing Rate (BR), the Average Breathing Rate (ABR) and the Breathing Rate Variability (BRV).

The Exhalation to Inhalation Ratio (EIR) may be calculated from the graph of FIG. 6a by measuring the Exhalation Duration (ED), the Inhale Duration (ID) and calculation EIR→ED/ID. Note that the breathing rate BR is given by 1/BD wherein the Breathing Duration BD=ED+ID. The values of EIR, BD, breathing depth and breathing stability may be assessed from the autocorrelation function, or from an FFT analysis or using other input devices such as a mobile phone or mouse as described below.

FIG. 6b shows a typical FFT spectrum of the HRV. An average breathing rate (ABR) can be inferred from the peak at around 1/10 Hz corresponding to average breathing cycle of 10 seconds. Average breathing depth may be inferred from the height of the peak and Variability in breathing rate from width of the peak.

By analyzing the FFT of the HR and analyzing the EDA over the same period, the balance of the sympathetic and parasympathetic nervous system can be analyzed.

Optionally or additionally, a conventional breathing sensor may be use to provide independent measurement of the breathing cycle. Optionally or additionally, the user may be requested to provide independent measure of the breathing cycle. For example, the user may be asked to use an input device of the mobile monitor, for example an LPT, [define], mouse or keypad, cellular phone keypad, scratchpad of a PDA or any other input device. The user may provide an input at each breathing cycle or provide more information, for example by pressing the “up” key during inhalation and the “down” during exhalation, thus providing information needed to calculate REI independently from the values inferred by HB analysis.

Alternatively or additionally, a microphone may be used as an input device to allow the user to speak an indication or the microphone is placed close to the user's airways to pick up noise caused by air currents during breathing. For example, a headset microphone attached to a cellular phone may be used for sensing the user's breathing. These methods are simple to implement, do not require a special respiration sensor, and provide important information and feedback to the user.

It was found that during relaxation, a breathing pattern is dominated by regular, slow, deep breathing. This pattern manifests itself by increased amplitude of the peak 60 in the curve of FIG. 6b. At the same time, due to the increased depth of inhalation, and the stabilization of the breathing rate, the Variability in HR increases, causing the broadening of the peak HRV shown in FIG. 6b.

Respiration Guide Bar

The system may present to the user a respiration guide using any one or more of a graphic bar display, musical cues voice instructions, and/or vibration. In the graphical bar display, the breathing bar length may vary, for example, in accordance with the user's respiration rate or the duration of the inspiratory or expiratory phase of the respiration cycle. The system can calculate the user's respiration rate and use it as a starting base line, and train him to improve the pace (increase the exhalation period) according to the user's needs, for example, using predetermined instructions that can be overridden by the user or a coach. As another example, the breathing bar length may vary in accordance with the lung volume of the user, increasing in length as the user inhales and decreasing as he exhales. Using an autocorrelation method, the application may anticipate the breathing pattern based on recent berating history. By displaying a delayed image of the breathing pattern, the user may train to slow down his breathing rate. Optionally, the training may be aimed at achieving a predetermined breathing rate goal. Similarly, the breathing depth, as determined by the HRV, may be indicted by the length of the breathing bar. Inspiratory and expiratory phases can easily be followed by the user observing the changing breathing bar. The speaker 126 may be used to give voice indications, encouragement and commands such as: “inhale”, “hold breath” or “exhale”. Alternatively, the breathing bar may change color according to the phase of the breathing cycle. Alternatively, another type of display, such as an expanding and contracting balloon may be displayed, where the size of the balloon represents the volume of the lungs. Optionally, the user may choose the operation and display mode of the breathing bar.

Display Screens

FIGS. 7a, 7b, 7c and 7d show exemplary display modes according to different embodiments of the invention.

It should be noted that these exemplary display screens are shown for demonstration purposes as adopted to be viewed on a specific cellular phone. Other display means, for example a PDA, etc, and display designs may be created within the general scope of the current invention.

FIG. 7a shows an exemplary display on a screen 122 of a cellular phone used as mobile monitor 120. On the top of the display screen 122 is an icon driven phone menu 72 that allows the user to access other functions of the cellular phone. In this example, the menu comprises: “incoming call” icon 73a, “address book” icon 73b, “message” icon 73c and it may comprise of other icons. At the bottom of the display screen 122 is a phone status line 86 showing status indicators of the cellular phone, such as “battery level” 81a, “speaker on” 81b, “RF reception level indicator” 81c, etc. Generally, these top and bottom lines are part of the cellular phone system and are not involved with the operation of the mobile unit as physiological monitoring and training.

Some or all functions of the mobile unit, for example cellular phone 120, are available to the user during physiological monitoring. For example, the user may accept an incoming call on the cellular unit. Preferably, physiological data continue to be accepted and logged, to be processed and displayed later. Similarly, the user may access an address book or other information stored in memory of the mobile unit without interruption of physiological data logging.

In the case where the mobile unit 120 is a cellular phone, the data analysis and screen display may be created by an application loaded into the cellular phone memory and executed by the processor within the cellular phone.

The data logged on the mobile unit may be transmitted to a remote server for further analysis. For example data may be sent to via the cellular network using a data exchange protocol such as GSM, GPRS or 3 G. Alternatively or additionally, data may be transferred to a PC or a laptop computer using a cable such as USB cable, Bluetooth RF communication or Infrared (IR) communication.

Below the icon driven phone menu 72 is an application menu 85 that allows the user to access other functions and display modes of the current invention. For example, the user can choose specific tutorial or interactive training. The application menu 75 may allow control of the sensor's mode of operation, for example: starting and stopping data acquisition or data transfer, turning on or off a sensor, determining the sampling rate and accuracy, etc. The user may use the application menu 85 to choose the format of the displayed graphs and data.

The display screen 122 may display breathing bar 77. In the examples herein, breathing bar 77 is in the upper left, below the application menu 75. In the embodiment of FIG. 7a, the graph 80 shows the pulse signal 81 plotted vs. time on the horizontal axis, as measured for example by blood flow in the skin which is monitored by Heart Rate (HR) Electronics 320 within sensor module 210. Preferably, the graph is continuously updated and displays the data in real time. Alternatively, the graph is represents previously logged data.

In the embodiment of FIG. 7a, the graph 90 shows the EDA signal 91 plotted vs. time on the horizontal axis, as measured by the EDA electronics 330 within the sensor module 210. Preferably, the graph is continuously updated and displays the data in real time. Alternatively, said graph may display previously logged data.

The large main graph 50 shows instantaneous HR(t) 51 in units of heart-beats per second on the vertical axis plotted vs. time in minutes on the horizontal axis. Optionally the main graph 50 comprises a navigation icon 54 (shown here in “play” state) used to manipulate the display. For example, the user can “freeze” the display to closely examine a specific time frame. Similarly, the user can perform any or all of the commands “fast forward,” “shift up”, “shift down”, “move back”, “zoom in”, “zoom out”, “smooth” etc. Manipulations performed on the large graph 50 may also effect one or both of the graphs 80 and 90 so as to maintain the synchronization of all the graphs. Alternatively, some of the graphs may show real time data while another graph shows previously logged data.

Target or optimal range zone limits 52a and 52b are marked on the main graph 50 so that the user can easily compare his heart rate to a training goal. The target zone may be colored. For example a central green zone may indicate the goal values, while shades of yellow designate the target zone and shades of red indicate dangerously high or low values. The background color of one or some of the graph may be indicative of the state of mind of the user.

In the embodiment of FIG. 7a, the numerical data on the left 65a shows the instantaneous heart rate HR(t). In this example, the value 61 beats per seconds may also be inferred from the last value of graph 51: Alternatively, numerical data on the left 65a may display the average heart rate over a predetermined time interval.

In the Embodiment of FIG. 7a, the numerical data on the right 65b shows the average heart rate variability as computed from the standard deviation of HR(t) over a time window. Alternatively, numerical data on the right 65b may display data indicative of the difference between the minimum heart rate and maximum heart rate as depicted in FIG. 6a.

FIG. 7b shows another exemplary display on a screen 122 of a cellular phone used as mobile monitor. In this example, graph 90 shows HRV values 93 plotted vs. time on the horizontal axis instead of showing EDA data. The values 93 may be indicative of an autocorrelation function of the HRV.

FIG. 7c shows another exemplary display on a screen of a cellular phone used as mobile monitor. In this embodiment, graph 90 shows HRV values 93 while large graph 50 shows EDA data 91. A navigation icon 54 indicates that the data display is in a “pause” mode.

FIG. 7d shows yet another exemplary display on a screen of a cellular phone used as mobile monitor. In this embodiment, graph 80 shows pulse data 81, graph 90 shows data 51 and graph 50 shows HRV data 93.

The exemplary screens depicted in FIGS. 7a to 7d may be used by a user to assess his physiological state and as a biofeedback device to modify his condition and reactions to daily events. The mobile monitor may be used to display “real-time” parameters calculated from data recently acquired or may be used to replay a sequence of parameters previously acquired and stored. The date and time at which the data were acquired may be stored and associated with the stored data and is optionally displayed too.

The display screens may be flexibly designed to fit the size and type of display of the mobile monitor. Different combinations of signals and parameters may be displayed in various ways such as graphs, colors, pie charts, numerical values, bars, clock-like indicators, alert signals, alphanumerical messages, etc. Static or moving animations may also be displayed according to the interpretation of the physiological data. For example, a happy “smiley face” may be displayed when the state of the user is relaxed and sad face when the user is in a state of anxiety. The speed of the motion of the animation may be correlated with vital parameters such as HR or BR. A pulsing heart or breathing lungs may be displayed and animated to follow the cycles of the user. Music and musical tones may also be used as indicators, for example the pitch or intensity may be correlated with HR and BR and the user may train to achieve and maintain low quiet sound.

Training Session

Because the EDA sensor as described herein is sensitive to changes in the arousal level of the user, it is possible to calculate several types of scores that reflect changes in the user's responses to different stimuli, including subconscious responses. The stimulus can be, for example, a question, a picture, music, a smell, or multimedia clips such as a short video. The stimulus can be presented/asked by another person or by prerecorded information on the mobile monitor or computer. It can be a message transmitted to the user such as text message or multimedia message on the mobile phone or TV clip or any other stimulus that can affect the user's response consciously or sub-consciously. The system monitors the user's physiology before, during and after the stimulus, and may calculate any one or more of the following parameters: EDA scores, heart scores and state of mind scores.

FIG. 8 shows an EDA graph as an example of a stress response of a user to such a stimulus. From these responses the system can calculate the following scores: the stimulus (trigger) time, the latency (response time) until the EDA changed, the time to maximum conductivity, the absolute and relative changes in the amplitude before the stimulus (baseline), during the stimulus, and the new base line after a predetermined time following the stimulus, the half recovery time, the full recovery time; the variance and standard deviation of the EDA calculated periodically (such as every one tenth of a second) before during and after the stimulus; calculating a similar parameter based on the variance of the EDA—including the standard deviation and/or variance of the variance of the EDA, and latency, maximum of the variance, half recovery time of the variance, and recovery time of the variance.

Trying these scores with many users, it was found that this system can be effective in finding which number a person has chosen or if he is or is not telling the truth, and detecting other information that the user tried to hide. For example, users were asked to choose a number. The mobile phone presents a randomly chosen number, and calculates the parameters described above. The user is instructed to say no to all the numbers. But the system can detect the number that the user had chosen by finding the number with the maximum standard deviation of the variance of the EDA after presenting the chosen number.

In a similar way the system also calculated changes in the pulse, heart rate and heart rate variability of the user during a specific time interval or as a response to a stimulus (heart scores).

In the exemplary embodiment of the invention, the system can monitor and calculate both EDA scores and pulse scores, and present to at least one user a multimedia audio-visual response on the mobile monitor. Therefore it is possible to present different audiovisual clips which represent different moods. The system can also record the user's subjective responses (degree of fear or joy) and calculate the EDA scores and the heart scores simultaneously. This can be used for research, for therapy, for assessment, and for fun. Using these methods it is possible to map at least two dimensions of a user's state of mind; one dimension is arousal or relaxation, and the second dimension is positive or negative—does the user enjoy this state or dislike it. FIG. 4 shows a two-dimensional array of states of mind. The present invention can be used to map an individual's state of mind in the two-dimensional array.

An additional aspect of the present invention is integration of Computerized Cognitive Behavioral Therapy (CCBT) together with the system of the invention (the sensors, algorithms as described). Several systems have been developed for computerized psychological methods known as CBT. For example, in a Doctorate thesis in Clinical Psychology August 2002, Kings College London UK Dr. Gili Orbach presented a Computerized Cognitive Behavior Therapy (CCBT) program. This is a method and clinical process to train students using a multimedia interactive program over the internet to reduce anxiety, and improve self confidence and results in exams. The CCBT programs can educate the users, explain to them about their thought mistakes, provide them with behavioral advice, etc. By integrating together CCBT, visualization, self hypnosis, and the present invention, including sensors and methods to monitor responses, and interactive multimedia feedback to train them to change their responses, a method and system are created, that can train users to modify their behavioral responses, know themselves better, help them to overcome habits and change themselves in their preferred direction.

Possible Uses

When the system of the present invention may be equipped with programmable data processing power and flexible output means, numerous applications and uses may be adopted and used, optionally simultaneously and in combinations. A few exemplary applications will be described below.

Alerts

The system may be programmed to alert the user or someone else when certain conditions occur. Conditions may be assessed, and an alert initiated by any or few of: processor 340 in the sensor module, in the mobile monitor 120, in the server 140 or by the human expert 150.

The system of the invention may generate an alert under predetermined conditions. Heart and breathing alerts may be life saving for patients at risk of heart attack, epilepsy, old or incapacitated people, people with mental disability etc. Alerts may be indicated by any or few of: indicator 380, display 120 and speaker 126. Alternatively or additionally, alerts may be relayed to other locations by any or few of: mobile monitor 120, server 140 or by the human expert 150. For example a medical, law enforcement or rescue team may be informed if the system detects possible behavior abnormality. Data supporting the assessment may be relayed in association with the alert. If it exists, data on identity, health condition such as medical records, and location of the user, for example a GPS reading of the mobile monitor, may also be transferred. Conditions for generating an alert may be related to heart rate for example: HR below or above a predetermined value, abnormal HRV for example HRV below or above a predetermined value or rapidly changing, or indication for arrhythmia. Conditions for alerts may be related to breathing for example: any or more of: HR, BR or ERI below or above a predetermined value, abnormal BR for example BR rapidly changing. Conditions for generating an alert may be related to stress for example: EDA below or above a predetermined value or rapidly changing. Conditions for generating an alert may be related to a combination of signals from multiple sensors.

Training for Improving Quality of Life

The system of the invention may be used for training aimed at modifying his condition. For example, the user may observe his physiological signs and optionally or alternatively the interpretation of these signs to modify his behavior to avoid negative emotions depicted on the right side of FIG. 4. Additionally, the user may train to achieve, strengthen or maintain concentration and enthusiasm depicted in the upper-left quadrant of FIG. 4 by modifying his behavior. Or, the user may train to achieve, strengthen or maintain a state of relaxation as depicted in the lower-left quadrant of FIG. 4.

It has been shown that people are able to achieve these goals by using biofeedback, even though they are not fully aware how they control their emotional and physical states, and thus gain control over involuntarily body activities such as blood pressure, hormone secretion etc. The system of the invention may also be used for training voluntary activity. For example a user may train to breath at a steady slow rate optionally achieving deep breathing with low ERI. This type of breathing is known to promote relaxation.

According to another embodiment of the invention, a user known to suffer from episodes of anger or anxiety may use the system in his daily routine. The system may be used to detect early signs of an approaching attack and prompt the user to take measures to mitigate the situation ether by taking medication or by mental or physical exercises such as taking deep breaths or by stopping his current activity. A silent alert such as vibration or a concealed alert such as Short Message Service SMS or a “fake” call to a cellular phone may serve to distract the user from the harmful path that may lead to aggressive or an anxiety attack. People suffering from various phobias may also benefit from an alert generated when a stimulus eliciting the phobia is approaching.

When the breathing cycle is followed by both HRV analysis and another means such as breathing sensor or user input, the correlation between HRV and actual breathing cycle may be monitored and the user may train to achieve better synchronization between the two. Generally, inhaling induces sympathetic system response causing arousal and increase of HR while exhaling induces the parasympathetic system response causing relaxation and decrease of HR. Thus, learning to control breathing, an art that currently requires years of studying, meditation or Yoga, may be achieved using the present invention.

According to another embodiment of the invention, the system may be used to record the physical and mental state of the user during his daily routine and correlate its readings to the type of activities performed. For example, times of high stress, high concentration, best performance, or high pleasure may be timed and displayed. The user may compare these times with the activities performed that date, for example, by referring to his diary records. Sensor readings may be integrated with diary records automatically, for example by integrating the software with commercial applications such as Microsoft Outlook®, and displayed on a mobile monitor such as a PDA or LPC.

Additionally or alternatively, the user may use input means on the mobile monitor to input memorandums such as voice or written messages indicating the type of activity he is performing, and his subjective feelings which will be integrated into the log of daily activity and sensor readings. In this way, the user may compare his activities and his subjective feelings to the objective sensor reading. Knowing the activities that induce stress, the user may prepare himself for future repetitions of the same or similar activities, or attempt to avoid them.

According to another embodiment of the invention, the system may be used to record physiological readings during sports training. In contrast to available devices that display only moving AHR, the system of the invention is capable of recording and storing virtually a record of each individual heartbeat and breath. Data compression, large memory capacity in the mobile monitor and mass storage in the remote server enable acquiring and storing these records over long periods of use. Because the sensors are small and transmit the data wirelessly—either using the Bluetooth protocol or the mobile network communication services—an expert coach can view and monitor the physiological parameters, the emotional-arousal states and the performance of the athlete, and coach him in real time to improve his reactions and performance. The data can be also saved for analysis later on. An athlete can also rehearse at his home or office using the invention, with either a multimedia mobile phone or PC or PDA (personal digital device) while he is viewing his performance, and simulating his emotional and physiological conditions, as in a real competition. By using several of the sensors simultaneously (e.g. heart rate, HRV, breathing, EDA EMG), the user learns to tune not only his physiology but also his attitude, arousal level etc, and to achieve his best performance.

According to another embodiment of the invention, the system may be used to record physiological reading while the user is sleeping in order to help identify and possibly correct sleep disorders.

Wearable Biofeedback Tools:

Biofeedback has been in use for many years to alleviate and change an individual's negative behavior patterns but existing systems have a number of significant drawbacks:

1. Hardware, software and information gathering:
Most current systems are reliant upon powerful computers
They require users to be trained either by health professionals or complex on-line programmers;
Once users have been trained they must remember to implement the internal physiological changes in their daily lives;
The biofeedback sessions are rarely undertaken on a daily basis and not in real time. This requires the user to remember specific events that occurred days before and recall his exact emotional responses.

This invention utilizes portable, cordless wearable sensors, which enable users to monitor their emotional and physiological responses to events as they occur. These results, gathered in real time, may be more effective and relevant to the user than those recreated days later under completely different conditions. The sensors of the invention utilize mobile phones to display the user's physiology and emotional state.

2. Methodology:

The current method is to train users to modify the underlying physiology related to negative behavior patterns for example, to reduce muscular tension (EMG), GSR, or electro-dermal activity (EDA)—the main purpose of which is to train users to relax. However, although it is important to train users to relax, two other aspects must also be taken into account for successful treatment:
Enhancement of emotional health and training to be more positive, enthusiastic and motivated. These states are not reflected in relaxation levels as measured by GSR, EDA or EMG which can give false impressions. For example, a user may display increased physical tension when experiencing positive emotions such as excitement or enthusiasm. Similarly, low levels of physical tension may not necessarily be a positive thing and could represent negative states such as depression or boredom. One example was use of EDA for people suffering IBS (irritable bowel syndrome). EDA was found to be very useful for people with high anxiety suffering from diarrhea, but not for depressed people suffered from constipation.

By utilizing two sensors simultaneously, a sensitive EDA sensor and a heart rate monitor for HRV, and by analyzing the changes in specific situations, the system of the invention may be used to monitor and train users not only to relax but also to develop a positive state of mind.

Objective Emotional Monitor:

Another application of the present invention is to monitor emotional reactions by using an objective scale. Although EDA is very sensitive there are disadvantages in monitoring and analyzing emotional reactions using this method:
EDA levels change between sessions and individuals because of many variables unrelated to a user's emotional state. Therefore EDA levels can only be interpreted as a trend. That is, the user is becoming more relaxed if his skin resistance is increasing above the level when the session began. But the user cannot learn in an objective way how to control his reactions and improve his physiology and performance. The sensor of the invention allows monitoring and real time presentation of changes related to thought and emotion and calculation of parameters that reflect how the user is responding to specific trigger events. By integrating the analysis of the change in the EDA and the Heart Rate and heart rate variability in real-time a scale can be created to enable the user to learn how to improve and monitor his reactions.

FIG. 8 shows response to a stimulus (such as PTSD, bulling, phobia). The parameters relating to the response include the amount of time it takes for the user to return to the base line after the stimulus, the amount of time it takes to return to baseline plus half arousal jump, the level of the arousal jump related to specific triggers. By using a mobile sensor, the user can continually monitor and improve his reactions and performance. By adding multimedia instructions the system can be a real time coach for the user. By transmitting the data in real time using a mobile phone user will be able:
To get feedback from a sophisticated expert system on a server almost in real time.
to record their reactions to specific situations during the day
To receive advice from an expert who can monitor their reaction almost in real time.
to modify their reaction and implement this new knowledge in their daily behavior while an expert (system or professional caregiver) monitors them.

Integrating CBT and a Wearable Bio Interactive Sensor

Existing biofeedback systems use behavioral methods but do not include CBT (Cognitive Behavioral Therapy) training. The system of the invention may integrate computerized CBT, visualization with interactive sensors allowing users to learn not only how to change their physiology but also modify their way of thinking and address negative thought patterns.

New Methods of integrated CEBIT (Cognitive Emotional Behavioral Interactive Therapy). Training utilizing an integrated sensor of the invention allows a user to examine his belief system, his behavior, his unconscious thought processes, emotional and cognitive reactions, and his physiology. It also trains the user to monitor himself, to be aware, listen to his body, his emotions, and his external reactions.

Performance improvement—by using the methods and systems of the invention, and by monitoring their progress, users can learn not only how to modify their health and feel better but also to improve their performance: e.g. exam anxiety, trading, music and singing, sports, relationships, creativity, public speaking etc. The interactive physiology monitoring of the invention can be combined with CBT, and with realtime feedback from the user's performance, to train the user to achieve a predetermined state. This can be applied also to relationships and to happiness level.

Survey and Poles

According to another application of the invention, the system may be used to record reactions of viewers to commercials in order to conduct viewer surveys.

Training Session

Yet another aspect of the invention is to train a user by conducting a training session involving exposing the user to stress inducing stimuli.

FIG. 8 shows a schematic chart of the stress level of a user following a stimulus The stimulus may be, for example, a phobia caused by an image, for example a picture of a spider to a user suffers from arachnophobia, a disturbing voice message or written phrase. Stress induced by the stimuli may be measured by EDA reading, HR, or a combination of few sensors readings.

In FIG. 8, the stimulus is given at time ST. At time LT, stress level starts to rise from the Initial Baseline Stress (IBS) after a short latency period in which the user's brain interprets the stimulus. Usually the stress climbs and reaches its Maximum Stress (MS) level at Maximum Reaction Time (MRT), then recovers slowly to the IBS or to a New Baseline Stress (NBS).

Recovery Time (RT) may be defined as the time it takes for the stress level to decrease from MS level to the Half maximum Stress (HS) at the Half Recovery Time (HRT), i.e. RT=HRT−MRT, where HS is defined as: HS=(IBS+MS)/2. In a training session, the user observes his reactions and learns to minimize one or more of MS, RT and NBS.

A training session may consist of analyzing HR, HRV and changes in EDA using several methods such as neural network software and or wavelet analysis, while presenting to the user specific positive and negative triggers. For example images, video or audio clips. Scenes such as of an accident may be used as negative triggers; while relaxing triggers may be nature scenes. Training may be in a form of interactive games in which the user can win and feel positive; frustrating games or challenges in which the user looses and feels stressed; sexual clips etc;

A “User psycho-physiological responses profile” (UPPP) may be created and stored. Using this UPPP, the system can monitor and analyze the user response and state of mind to both real life events (e.g. a meeting with someone, preparing for an exam, receiving a phone call, etc), and or interactive questionnaires, simulation of specific scenarios, etc. These methods can be used for several purposes: to assess the user responses and/or to train the user to improve his responses to specific triggers (such as overcoming a phobia). The system can use the UPPP to drive games and multimedia using the sensors and the user's emotional reaction to drive and navigate the games.

The term “user” should be interpreted as encompassing both a male and a female individual, and also to a group of individuals. When there are several users, each one can be monitored with his sensors, or some of them can share sensors, they can either use the same display (for example connected with Bluetooth to the same PC or mobile phone) or each one can have a separate device with their devices configured to communicate with each other. It can also include a plurality users connected through mobile phones or Internet to a center or TV station, watching and sharing one or more images which are transmitted either as broadcast or internet etc to all the users or some of them. In this mode the invention can be used as a new real-time TV show game, or emotional poll, etc.

Entertainment System: Mind Activated Games for Interactive Communication

According to another aspect of the invention, the system may be used for entertainment by providing games and other forms of entertainment.

For example, a person may use the sensor module during a phone conversation or Internet chat with peers. The sensor readings may automatically send SMS or pictorial symbols indicating the user's state of mind and his reactions to the conversation. This can be a basis for emotional based games and communication between a group of users of mobile phones and/or internet and or TV games.

In another example, sensor readings may be used to control devices and appliances such as a DVD or compute, for example, during computer games. sensor can be added to a remote control, and the content presented to the users can be changed and unfold according to the state of mind of the users who are monitored by the sensors. This can be a basis for a new interactive DVD (or any alternative direct access digital media), for interactive movies, interactive sport, or interactive games, or psychological profiling.

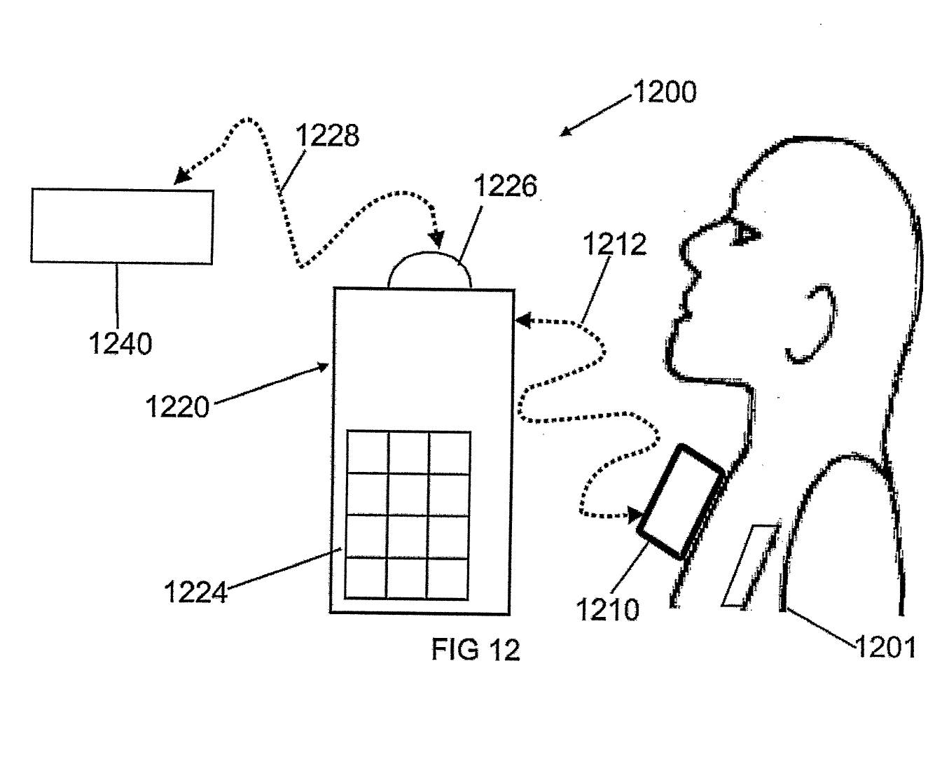

FIG. 12 depicts an entertainment system 1200 according to one embodiment of this aspect of the invention. In the system 1200, a sensor 1210 is in contact with a user 1201 and is used for monitoring the user's physiological parameters. The Sensor 1210 is in communication with an entertainment system controller 1220, such as a remote control of a DVD or video game device, through communication link 1212. Communication link 1212 may be unidirectional or bi-directional. The entertainment system controller 1220 comprises a transmitter 1226 for transmitting commands to the entertainment system 1240 using communication link 1228. Link 1228 may be unidirectional, for example, IR communication. Optionally, the system controller 1220 comprises of an input means such as keypad 1224. The sensor 1210 may directly communicate with the entertainment system, and a cable may be used for communicating physiological information or commands.

In accordance with this aspect of the invention, at least one parameter reflecting a state of mind and or body of the players/users is obtained by monitoring one or more parameters indicative of their physiological or psycho-physiological reactions/conditions. The one or more parameters are transmitted to a system that analyses the parameters and calculates one or more scores and uses the calculated scores as input for a process in which audiovisual material (audio and/or visual) is displayed on a screen and/or a physical object (such as remote controlled car) is moved. The content of the audiovisual material, and/or some of the parameters of the movement (e.g. the speed or direction of movement of the remote controlled car) depend on the scores reflecting the state of the user's mind and or body.

The scores, or some information which reflect results of changes in the state of mind and or body of the user or users may be presented directly or indirectly either to the same user/player that is being monitored by the sensor or to another user/player or to both of them. The users may use information relating to either their own scores/results or the other players' scores/results in order to win or change their reactions/decisions or to guess the other user's feelings or thoughts, or to influence the other user's reactions, or the results of the games/interactive story/remote controlled toy.

Examples of Games
Battleship (submarines). In this a familiar game, two players try to guess and find the location of the opponent player's submarines/ships and “destroy” them, (for example in a 10 by ten array of positions). The present invention may be used to add a new aspect to the game. Before user A “shoots” a torpedo to a specific location (the location “b-4”, for example) he can ask the other player 3 questions (e.g. by words or by moving a mouse to specific locations but not clicking it). The questions may be, for example “Do you have submarine in location b-2 or b-4 or c-4?”. The user A can see the reaction of the other player as reflected in one of his scores. The other user can respond yes or not and can even lie (high arousal—high bar). User A can use this information to assess where there is a submarine. Thus, a psychological and “mind reading” dimension is added to a game.
   a) A group of users, such as teenagers, with mobile phones can send multimedia messages to each other and view pictures and/or a short video of each other. Using this invention we add an emotional dimension to the communication as follows. The scores of the emotional and/or state of mind reaction are also transmitted to the other users, and these scores are used as a basis for games and interactive communication, such as a truth or dare game. The reaction (emotional scores) of a user is transmitted to one or more other users. For example, the scores may be sent to a first user that was the most “aroused” when he or she saw the picture and/or read an MMS message from a particular second user. The first user then has to send a text message to the second user revealing what the first user feels about the second user. While the first user does this, the first user's arousal level can be watched by the second user and/or other users. Thus, either the “system” and or other users and/or the first user can see if the first user “loves” the second user. In a simple version of this game, a user can see 10 pictures on the screen of his mobile phone or PC or game console and the system can tell him, for example, who he loves, which number he has chosen, or which card he has chosen.
   b) Interactive “Tamaguchi” (an electronic pet or animation of a person which the user has to “love” and take care). By incorporating the features of the present invention to this toy, each time that the user is angry and/or anxious, as indicated by the scores obtained from the results monitored by the sensors, the Tamaguchi can feel it and react, be sad, angry, or ill, etc. When the user is calm, relaxed and happy, the Tamaguchi reacts in a positive way, e.g. by smiling, singing, playing, eating etc.
   c) In a more advanced version, a user can create a symbolic animated version of himself (a “virtual me” or “Vime”) in a mobile phone, PC or game consol. The user and/or other individuals (that have received permission/authority to interact with the user's virtual personality), can interact with this “Virtual me” using a mobile communication device or Internet. An individual may play with the user's virtual personality, for example, by sending the Vime positive and/or negative messages such as that the individual loves the Vime. The “conscious” message is transmitted together with the individual's State of Mind/emotional score and influences the “virtual me”. This can be used as games and entertainment but also as adding an emotional dimension and new way of communication and playing, and even virtual “dating”.
   d) Behavioral skills may be added to the version of the game presented in c) such as how to react and with whom. This can create a psychological/emotional/communication game/community creation. For example, real or imaginary qualities can be added to the Vime and descriptions (physical dimensions, hobbies, area of interest etc); behavioral rules (“if a girl with predetermined characteristics and predetermined scores contacts me then send a predetermined response”). The Vime can have several modes such as a “live” mode in which the user is connected, an “offline” mode in which the Vime can communicate without the user, a “receive only” mode, or a “sleep” mode.
   e) In another application, the sensors are used as amplifiers of subconscious intuition responses, for example to provide real or fun decision advice. While the user is connected to the sensors, he asks questions and/or is asked questions by the phone, PC or DVD. By watching his scores when he thinks and answers a specific question he can see what his “intuition” advises him to do. The system may train the user to tune himself to make a better decision by integration of his or her physiological and psychological states, together with other methods such as logical analysis, systematic planning, scoring etc. (i.e. “to use his heart and his brain” together, or to use his analytical mind with his intuition, to combine his “gutfeelings” with “objective information?”).

While the invention has been described with reference to certain exemplary embodiments, various modifications will be readily apparent to and may be readily accomplished by persons skilled in the art without departing from the spirit and scope of the above teachings.

It should be understood that features and/or steps described with respect to one embodiment may be used with other embodiments and that not all embodiments of the invention have all of the features and/or steps shown in a particular figure or described with respect to one of the embodiments. Variations of embodiments described will occur to persons of the art.

It is noted that some of the above described embodiments may describe the best mode contemplated by the inventors and therefore include structure, acts or details of structures and acts that may not be essential to the invention and which are described as examples. Structure and acts described herein are replaceable by equivalents which perform the same function, even if the structure or acts are different, as known in the art. Therefore, the scope of the invention is limited only by the elements and limitations as used in the claims. The terms “comprise”, “include” and their conjugates as used herein mean “include but are not necessarily limited to”.

1. A system for monitoring one or more physiological parameters of a user comprising:
(a) one or more wearable sensor modules sensing the one or more physiological parameters;
(b) one or more transmitters wirelessly transmitting first signals indicative of values of the one or more physiological parameters to a mobile monitor; and
(c) the mobile monitor, wherein the mobile monitor comprises:
a first processor processing the first signals received from the transmitter in real time using expert knowledge; and
a device providing one or more indications of results of the processing.

2. The system according to claim 1 further comprising a remote server capable of communication with said mobile monitor, the remote server receiving second signals from the mobile monitor, the remote server associated with a viewing station having a second processor, the remote server being configured to perform at least one of the following:
(a) transmitting the second signals to a viewing station for analysis, the analysis;
(b) accessing historical data relating to the subject;
(c) transmitting the historical data to the viewing station;
(d) receiving from the viewing station results of the analysis;
(e) transmitting the results of the analysis to the mobile unit; the analysis being based upon the second signals, and one or more of the historical data, expert knowledge and computerised protocols.

3. The system according to claim 1 wherein at least one sensor module comprises at least one sensor selected from the group comprising:
(a) An electro dermal activity sensor;
(b) An electrocardiogram sensor;
(c) A plethysmograph; and
(d) A piezoelectric sensor.

4. The system according to claim 1 comprising at least two sensors selected from a group comprising:
(a) an electro dermal activity sensor;
(b) an electrocardiogram sensor;
(c) a plethysmograph; and
(d) a respiration sensor.

5. The system according to claim 1 wherein the first signals are transmitted from a sensor module to the mobile monitor by any one or more of the following protocols:
(a) Bluetooth;
(b) WiFi; and
(c) Wireless Lan;

6. The system according to claim 1 wherein said mobile monitor is selected from the group comprising:
(a) a cellular phone;
(b) a personal digital assistant (PDA);
(c) a pocket PC;
(d) a mobile audio digital player;
(e) an iPod,
(f) an electronic note-book;
(g) a personal laptop computer;
(h) a DVD player;
(i) a hand held video game with wireless communication; and
(j) mobile TV.

7. The system according to claim 6 wherein the mobile unit is a cellular telephone and communication between the mobile monitor and the remote server is over a cellular communication network.

8. The system according to claim 1 wherein the mobile unit includes any one or more of a visual display, one or more speakers, a headphone, and a virtual reality headset.

9. A wearable sensor module for use in the system according to claim 1.

10. The wearable sensor module according to claim 9 comprising at least one sensor selected from the group comprising:
(a) An electro dermal activity sensor;
(b) An electrocardiogram sensor;
(c) A plethysmograph; and
(d) A pizoomagnetic sensor.

11. The wearable sensor module according to claim 10 comprising at least two sensors selected from a group comprising:
(a) an electro dermal activity sensor;
(b) an electrocardiogram sensor;
(c) a plethysmograph; and
(d) a respiration sensor.

12. The wearable sensor module according to claim 10 comprising a transmitter transmitting signals by any one or more of the following protocols:
(a) Bluetooth;
(b) WiFi; and
(c) Wireless Lan;

13. The wearable sensor unit according to claim 10 or 11 comprising an electro dermal activity sensor adapted to monitor skin conductivities using at least a 16 bit A to D conversion without the need of manual calibration.

14. The sensor module according to claim 10 or 11 comprising an EDA sensor comprising:
(a) at least two electrodes adapted to be applied to a skin surface;
(b) electronic circuitry for measuring a skin resistance across the electrodes and calculating an EDA based upon the resistance using an algorithm in which the EDA does not depend linearly on the resistance.

15. The sensor module according to claim 10 or 11 comprising a blood flow sensor comprising:
(a) a light source adapted to emit light towards a skin surface;
(b) a light detector adapted to detecting light reflected from the skin surface;
(c) electronic circuitry for measuring an intensity of the reflected light and controlling an intensity of said light source based upon the intensity of the reflected light.

16. The sensor module according to claim 14, wherein the electronic circuitry capable of measuring skin resistance across the electrodes over a range of at least from 50 K Ohm to 12 M Ohm.

17. The system according to claim 1 wherein the first processor is configured to calculated from the first signals one or both of a parameter indicative of an arousal state of the user and a parameter indicative of an emotional state of the user.

18. The system according to claim 14 wherein calculation of the parameter indicative of an arousal state of the user includes calculating a score of a sympathetic and parasympathetic activity of the user using an algorithm based on any one or more of the user's Electro Dennal activity, Heart Rate, EDA variability, and HR variability.

19. The system according to claim 14 wherein the first processor is configured to calculate a parameter indicative of an arousal state of the user to display the parameter indicative of an arousal state of the user on a display associated with the mobile unit as a two-dimensional vector.

20. The system according to claim 1 wherein the first processor is configured to display on a display associated with the mobile monitor any one or more of the following images: an image indicative of bio-feedback information relating to the user; an image indicative of breathing activity of the user, an image including a graph indicative of an EDA activity of the user, an image including a graph indicative of a heart rate of the user, an image including a graph indicative of a heart rate variability of the user; an image including a graph indicative of an autocorrelation of a heart rate variability of the user; and an image indicative of recommendation to improve the user's psycho-physiological state based on one or both of the user's physiological data and experts' knowledge.

21. The system according to claim 17 wherein an image indicative of breathing activity includes a bar having a length indicative of the breathing activity.

22. The system according to claim 17 wherein an image indicative of bio-feedback information relating to the user includes one or more parameter target values.

23. The system according to claim 1 wherein the first processor is configured to calculate in a calculation based upon the first signals any one or more of the following: a breathing rate of the user; and a heart rate variability of the user.

24. A system according to claims 23 wherein the user's rate of breathing is calculated and analysis by monitoring changes in the electrical capacitance of the body while the user is breathing.

25. A method for monitoring one or more physiological parameters of a user comprising:
(a) obtaining values of the physiological parameters of the user from one or more wearable sensor modules;
(b) wirelessly transmitting first signals indicative of values of the one or more physiological parameters to a mobile monitor; and
(c) processing the first signals received from the transmitter in real time using expert knowledge; and
(d) providing one or more indications of results of the processing to the mobile unit.

26. The method according to claim 25 wherein the results of the processing includes bio-feedback information of the user.

27. The method according to claim 25 further comprising transmitting second signals from the mobile monitor to a remote server having an associated viewing station and providing an analysis of the second signals at the viewing station.

28. The method according to claim 27 wherein the viewing station includes one or both of a remote call center and an interactive expert system.

29. The method according to claim 25 wherein the processing includes calculating one or both of a parameter indicative of an arousal state and a parameter indicative of an emotional state of the user.

30. The method according to claim 29 wherein calculating a parameter indicative of an emotional state of the user is based upon one or both of a sympathetic activity and parasympathetic activity of the user.

31. The method according to claim 30 wherein calculating a parameter indicative of an emotional state of the user is based upon any one or more of an electro dermal activity, a heart rate, an electro dermal activity variability and a heart rate variability.

32. The method according to claim 29 further comprising the step of displaying on a display associated with the mobile unit one or both of an image indicative of a parameter indicative of an arousal state of the user; and an image indicative of a parameter indicative of emotional state of the user.

33. The method according to claims 32 wherein an image includes one or both of a two-dimensional vector and a color indicative of a parameter.

34. The method according to claim 25 for use in obtaining respiration information selected from the group comprising duration of the inspiratory phase, and duration of the expiratory phase.

35. A method according to claim 34 wherein respiratory information is obtained from audio sounds produced during breathing or speaking.

36. The method according to claim 34 wherein respiratory information is obtained by the user indicating the beginning of one or more inspiratory phases and the beginning of one or more expiratory phases of the user's breathing.

37. The method according to claim 34 wherein_a breathing rate of the user is calculated based upon a heart rate variability of the user.

38. The method according to claim 34 wherein the user's rate of breathing is calculated based upon changes in an electrical skin capacitance of the user while the user is breathing.

39. The method according to claim 34 further comprising training the user to increase any one or more of the followings: a duration of the inspiratory phase, a duration of the expiratory phase, and the ratio of the duration of the inspiratory phase to the duration of the expiratory phase.

40. The method according to claim 26, further comprising displaying on a display associated with the mobile monitor an image indicative of bio-feedback information, wherein the image includes any one or more of the following: an image indicative of breathing activity, an image including a graph indicative of EDA activity, an image including a graph indicative of heart rate, an image including a graph indicative of heart rate variability and an image including a graph indicative of an autocorrelation of heart rate variability.

41. The method according to claim 27 wherein the analysis of the second signals includes a recommendation for the user to improve a psycho physiological state of the user.

42. The method according to claim 41 further comprising displaying the recommendation on a display associated with the mobile unit.

43. The method according to claim 26 comprising displaying a target value for one or more of the one or more obtained physiological parameters.

44. The method according to claim 26 comprising displaying on a display associated with the mobile unit a target value for one or more of the one or more obtained physiological parameters.

45. The method according to claim 26 comprising steps of:
(a) challenging the user with one or more stimuli;
(b) monitoring one or more reactions of the user to said one or more stimuli;
(c) calculating, in a calculation based upon the one or more reactions, at least one parameter selected from the group of: latency time of a reaction, maximum reaction time, half recovery time, maximum stress, and new baseline stress; and
(d) providing feedback to the user based on one or more of the calculated parameters.

46. The method according to claim 25 for use in a method of self behaviour modification comprising any one or more of the methods selected from the group comprising:
(a) cognitive behavioural therapy (CBT);
(b) visualisation;
(c) self hypnosis;
(d) auto suggestion;
(e) mindfulness;
(f) meditation;
(g) emotional intelligence skills;
(h) psychological counseling provided over a communications network.

47. The method according to claim 46 further comprising:
(a) providing the user with an interactive introduction about a specific condition of the user;
(b) providing the user interactive questionnaires for self assessment; and
(c) providing the user with one or more interactive sessions selected from the group comprising:
an interactive session for self training to implement cognitive techniques;
interactive sessions for self training to implement behavioural therapy;
interactive sessions for self hypnosis;
interactive sessions for visualisation;
interactive sessions for auto suggestions;
interactive training to acquire and implement life and interpersonal relational skills;
interactive training to improve emotional intelligence skills;
interactive training to find purposes and goals; and
interactive training to plan steps in life.

48. The method according to claim 47 wherein the user is provided with one or more interactive sessions while the user is in a deep relaxation state.

49. The system according to claim 1 further comprising an entertainment system and wherein the first processor is configured to determine at least one command based on the first signals and transmitting the at least one command based to the entertainment system; and wherein the entertainment system comprises a third processor configured to perform an action based upon the one or more commands.

50. The system according to claim 49 wherein the action comprises any one or more of generating an SMS massage, controlling a DVD, controlling a computer game, and controlling a “Tamaguchi” animation.

51. The system according to claim 49 wherein the action comprises processing a user reaction to any one or more of the following: a displayed animated image; a video clip, an audio clip, a multimedia presentation, real-time communication with another human, a question that the user has to answer, and a task that the has to perform.
